(19) United States
(12) Patent Application Publication
(10) Pub. No.: US 2024/0132538 A1
(43) Pub. Date: Apr. 25, 2024

(54) PROTEIN PURIFICATION USING A SPLIT INTEIN SYSTEM

(71) Applicant: Cytiva Bioprocess R&D AB, Uppsala (SE)

(72) Inventors: Christopher James Sevinsky, Niskayuna, NY (US); Peter Lundback, Uppsala (SE); Johan Ohman, Uppsala (SE); Gregory Grossmann, Niskayuna, NY (US); Sean R. Dinn, Niskayuna, NY (US)

(21) Appl. No.: 17/768,461

(22) PCT Filed: Nov. 20, 2020
(86) PCT No.: PCT/EP2020/082966

§ 371 Date: Apr. 12, 2022

(30) Foreign Application Priority Data
Nov. 22, 2019 (GB) 1917046.3

(51) Int. Cl. C07K 1/22; C07K 14/195; C07K 17/10

(52) U.S. Cl. CPC C07K 1/22; C07K 14/195; C07K 17/10; C07K 2319/92

(57) ABSTRACT

The present invention relates to protein purification, primarily in the chromatographic field. More closely, the invention relates to affinity chromatography using a split intein system with an improved C-intein tag and N-intein ligand, wherein the target protein may be purified as a tag-less end product with a native N-terminus.

### FIELD OF THE INVENTION

The present invention relates to protein purification, primarily in the chromatographic field. More closely, the invention relates to affinity chromatography using a split intein system with an improved C-intein tag and N-intein ligand, wherein the target protein may be purified as a tag-less end product with a native N-terminus.

### BACKGROUND OF THE INVENTION

Inteins are protein elements expressed as in-frame insertions that interrupt enzyme sequences and catalyze their own excision and ligation of two flanking polypeptides, generating an active protein. Genetically, inteins are encoded in two distinct ways: as intact inteins, interrupting two flanking extein sequences, or as split inteins, wherein each extein and part of the intein are encoded by two different genes. While they hold great promise as bioengineering and protein purification tools, split inteins with rapid kinetic properties found in nature are dependent on specific amino acids at the intein-extein junction, severely limiting the proteins that can be fused to inteins for affinity purification and recovery of native protein sequences. In particular, the prototypical split intein DNAE from Nostoc punctiforme exhibits kinetic properties suitable for protein purification applications. However, its activity is dependent on phenylalanine at the +2 position in the C-extein. This dependency severely narrows and impairs its general applicability.

Inteins have been engineered to accomplish several important functions in biotechnology, including applications as self-cleaving proteins for recombinant protein purification. Split inteins are particularly promising in this regard, as they can simultaneously provide affinity ligand and self-cleavage properties. In protein purification, a target protein that is the subject of purification may be substituted for either extein. To date, the DNAE family of split inteins has shown the most promise with C-terminal cleavage protein purification approaches.

WO2014/004336 describes proteins fused to split intein N-fragments and split intein C-fragments which could be attached to a support. The solid support could be a particle, bead, resin, or a slide.

WO2014/110393 describes proteins of interest fused to a split intein C-fragment which is contacted with a split intein N-fragment and a purification tag. The N-fragment may be attached to a solid phase via the purification tag and methods for affinity purification are discussed.

U.S. Pat. No. 10,066,027 describes a protein purification system and methods of using the system. Disclosed is a split intein comprising an N-terminal intein segment, which can be immobilized, and a C-terminal intein segment, which has the property of being self-cleaving, and which can be attached to a protein of interest The N-terminal intein segment is provided with a sensitivity enhancing motif which renders it more sensitive to extrinsic conditions.

U.S. Pat. No. 10,308,679 describes fusion proteins comprising an N-intein polypeptide and N-intein solubilization partner, and affinity matrices comprising such fusion proteins.

WO 2018/091424 describes a method for production of an affinity chromatography resin comprising an amino-terminal, (N-terminal), split intein fragment as an affinity ligand, comprising the following steps: a) expression of an N-terminal split intein fragment protein as insoluble protein in inclusion bodies in bacterial cells, preferably E. coli, b) harvesting said inclusion bodies; c) solubilizing said inclusion bodies and releasing expressed protein; d) binding said protein on a solid support; e) refolding said protein; f) releasing said protein from the solid support; and g) immobilizing said protein as ligands on a chromatography resin to form an affinity chromatography resin. This procedure enables immobilization a ligand density of 2-10 mg/ml resin.

As described above, split inteins have been used for protein purification using a combined affinity tag and tag cleavage mechanism. However, the utility of such systems, is limited by several factors. First, there is the amino acid requirements at the splice junction of the intended product, i.e. the requirement of Phe in the +2 position of the C-extein, to effect cleavage and attain purification of tag-less proteins. Recombinant protein production without extraneous amino acid on the N-terminus is highly desirable. Second, the protein releasing cleavage has to be sufficiently fast and provide an acceptable yield. Third, there is a solubility requirement of the split intein N- or C-fragment for attachment thereof to a solid support. Fourth, hitherto there are no available split intein systems suitable for large scale purification of tag-less proteins.

### SUMMARY OF THE INVENTION

The present invention overcomes the disadvantages within prior art and enables generic purification of tag-less/native proteins in just one rapid affinity chromatography step using a split intein system.

The present invention provides N-intein protein variant sequences of native split inteins or consensus sequences derived from native inteins and split inteins wherein, the N-intein variant is modified as compared to the native sequence or consensus sequence to eliminate all asparagine (N) amino acid residues present in the sequence. Preferably all such N-intein variant sequences are further modified to substitute cysteine (C) at position 1 with any other amino acid that is not cysteine.

The present invention provides N-intein protein variants of native split inteins or consensus sequences derived from inteins/split inteins wherein the N-intein protein variant does not include an asparagine (N) at position 36 of the variant sequence. This position is calculated according to conventional clustal alignment with native split inteins starting from the initial catalytical cysteine which is number 1. This position is conserved to N in prior art and native N-intein sequences but the present inventors have found that this position may be mutated to other amino acids that are less senstivie to deamidation such as histidine (H or His) or glutamine (Q or Gln), and to thereby achieve increased alkaline stability, which is important as it gives tolerance to increased pH values during for example chromatographic procedures. At least the N at position 36 has to be mutated, but it is also contemplated that more N may be mutated, preferably to H or Q, in the N-intein sequence.

The present invention also provides N- and C-inteins which overcome the absolute requirement of phenylalanine in the +2 position of the target protein of interest (POI). The N- and C-inteins of the invention can be used for production of any recombinant protein. By using the N- and C-inteins of the invention tag cleavage will occur at the exact junction of the tag intein and the POI, which means that the POI will be expressed in its native form with no extraneous amino acids encoded by the affinity tag. Furthermore, with the intein sequences of the invention, the POI is produced in high yield and with fast cleavage kinetics. The N-intein is coupled to solid phase which can be regenerated under alkali conditions.

The present invention provides an N-intein, a C-intein, a split intein system and methods of using the same as defined in the appended claims.

### BRIEF DESCRIPTION OF THE DRAWINGS

FIG. 1 is a graph showing the relative binding capacity for N-intein ligands according to the invention (A40, A41 and A48) coupled to an SPR biosensor chip.

FIG. 2 is a staple diagram showing the relative binding capacity for N-intein ligands according to the invention (B72, B22, A48) and a comparative ligand (A53) coupled to an SPR sensor chip.

FIG. 3 shows static binding capacity of the N-intein ligands of the invention. Amino acid analysis (AAA) is done by conventional method. A48 prototypes are coupled by epoxy chemistry to porous agarose particles.

FIG. 4A is a chromatogram of the purification results of Experiment 6.

FIG. 4B shows the SDS PAGE results from Experiment 6.

FIG. 5 is a graph showing the relative binding capacity for N-intein ligands according to the invention (A40 and A48) coupled to an SPR biosensor chip.

### DETAILED DESCRIPTION OF THE INVENTION

### Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

The term “contacting” as used herein refers to bringing two biological entities together in such a manner that the compound can affect the activity of the target, either directly; i.e., by interacting with the target itself, or indirectly; i.e., by interacting with another molecule, co-factor, factor, or protein on which the activity of the target is dependent. “Contacting” can also mean facilitating the interaction of two biological entities, such as peptides, to bond covalently or otherwise.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

The term “peptide”, “polypeptides” and “protein” are used interchangeably herein and include proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). Peptides include any oligopeptide, polypeptide, gene product, expression product, or protein. A peptide is comprised of consecutive amino acids and encompasses naturally occurring or synthetic molecules.

In addition, as used herein, the term “peptide” refers to amino acids joined to each other by peptide bonds or modified peptide bonds, e.g., peptide isosteres, etc. and may contain modified amino acids other than the 20 gene-encoded amino acids. The peptides can be modified by either natural processes, such as post-translational processing, or by chemical modification techniques which are well known in the art. Modifications can occur anywhere in the peptide, including the peptide backbone, the amino acid side-chains and the amino or carboxyl termini. The same type of modification can be present in the same or varying degrees at several sites in a given polypeptide. Also, a given peptide can have many types of modifications. Modifications include, without limitation, linkage of distinct domains or motifs, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, “variant” refers to a molecule that retains a biological activity that is the same or substantially similar to that of the original sequence. The variant may be from the same or different species or be a synthetic sequence based on a natural or prior molecule. Moreover, as used herein, “variant” refers to a molecule having a structure attained from the structure of a parent molecule (e.g., a protein or peptide disclosed herein) and whose structure or sequence is sufficiently similar to those disclosed herein that based upon that similarity, would be expected by one skilled in the art to exhibit the same or similar activities and utilities compared to the parent molecule. For example, substituting specific amino acids in a given peptide can yield a variant peptide with similar activity to the parent.

In the context of the present invention, a substitution in a variant protein is indicated as: [original amino acid/position in sequence/substituted amino acid] For example, an asparagine (N) at position 36 of an amino acid sequence that has been mutated to a histidine (H) is indicated interchangeably as “N36H” or “N36 to H”.

As used herein, the term “protein of interest (POI)” includes any synthetic or naturally occurring protein or peptide. The term therefore encompasses those compounds traditionally regarded as drugs, vaccines, and biopharmaceuticals including molecules such as proteins, peptides, and the like. Examples of therapeutic agents are described in well-known literature references such as the Merck Index (14th edition), the Physicians' Desk Reference (64th edition), and The Pharmacological Basis of Therapeutics (1st edition), and they include, without limitation, medicaments; substances used for the treatment, prevention, diagnosis, cure or mitigation of a disease or illness; substances that affect the structure or function of the body, or pro-drugs, which become biologically active or more active after they have been placed in a physiological environment.

As used herein, “isolated peptide” or “purified peptide” is meant to mean a peptide (or a fragment thereof) that is substantially free from the materials with which the peptide is normally associated in nature, or from the materials with which the peptide is associated in an artificial expression or production system, including but not limited to an expression host cell lysate, growth medium components, buffer components, cell culture supernatant, or components of a synthetic in vitro translation system. The peptides disclosed herein, or fragments thereof, can be obtained, for example, by extraction from a natural source (for example, a mammalian cell), by expression of a recombinant nucleic acid encoding the peptide (for example, in a cell or in a cell-free translation system), or by chemically synthesizing the peptide. In addition, peptide fragments may be obtained by any of these methods, or by cleaving full length proteins and/or peptides.

The word “or” as used herein means any one member of a particular list and also includes any combination of members of that list.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

As used herein, “isolated nucleic acid” or “purified nucleic acid” is meant to mean DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequences. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or peptide molecules.

As used herein, “extein” refers to the portion of an intein-modified protein that is not part of the intein and which can be spliced or cleaved upon excision of the intein.

“Intein” refers to an in-frame intervening sequence in a protein. An intein can catalyze its own excision from the protein through a post-translational protein splicing process to yield the free intein and a mature protein. An intein can also catalyze the cleavage of the intein-extein bond at either the intein N-terminus, or the intein C-terminus, or both of the intein-extein termini. As used herein, “intein” encompasses mini-inteins, modified or mutated inteins, and split inteins.

As used herein, the term “split intein” refers to any intein in which one or more peptide bond breaks exists between the N-terminal intein segment and the C-terminal intein segment such that the N-terminal and C-terminal intein segments become separate molecules that can non-covalently reassociate, or reconstitute, into an intein that is functional for splicing or cleaving reactions. Any catalytically active intein, or fragment thereof, may be used to derive a split intein for use in the systems and methods disclosed herein. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing splicing reactions.

As used herein, the “N-terminal intein segment” or “N-intein” refers to any intein sequence that comprises an N-terminal amino acid sequence that is functional for splicing and/or cleaving reactions when combined with a corresponding C-terminal intein segment. An N-terminal intein segment thus also comprises a sequence that is spliced out when splicing occurs. An N-terminal intein segment can comprise a sequence that is a modification of the N-terminal portion of a naturally occurring (native) intein sequence. Non-intein residues can also be genetically fused to intein segments to provide additional functionality, such as the ability to be affinity purified or to be covalently immobilized.

As used herein, the “C-terminal intein segment” or “C-intein” refers to any intein sequence that comprises a C-terminal amino acid sequence that is functional for splicing or cleaving reactions when combined with a corresponding N-terminal intein segment. In one aspect, the C-terminal intein segment comprises a sequence that is spliced out when splicing occurs. In another aspect, the C-terminal intein segment is cleaved from a peptide sequence fused to its C-terminus. The sequence which is cleaved from the C-terminal intein's C-terminus is referred to herein as a “protein of interest POI” is discussed in more detail below. A C-terminal intein segment can comprise a sequence that is a modification of the C-terminal portion of a naturally occurring (native) intein sequence. For example, a C terminal intein segment can comprise additional amino acid residues and/or mutated residues so long as the inclusion of such additional and/or mutated residues does not render the C-terminal intein segment non-functional for splicing or cleaving.

A consensus sequence is a sequence of DNA, RNA, or protein that represents aligned, related sequences. The consensus sequence of the related sequences can be defined in different ways, but is normally defined by the most common nucleotide(s) or amino acid residue(s) at each position. An example of a consensus sequence of the invention is the N-intein consensus sequence of SEQ ID NO: 6.

As used herein, the term “splice” or “splices” means to excise a central portion of a polypeptide to form two or more smaller polypeptide molecules. In some cases, splicing also includes the step of fusing together two or more of the smaller polypeptides to form a new polypeptide. Splicing can also refer to the joining of two polypeptides encoded on two separate gene products through the action of a split intein.

As used herein, the term “cleave” or “cleaves” means to divide a single polypeptide to form two or more smaller polypeptide molecules. In some cases, cleavage is mediated by the addition of an extrinsic endopeptidase, which is often referred to as “proteolytic cleavage”. In other cases, cleaving can be mediated by the intrinsic activity of one or both of the cleaved peptide sequences, which is often referred to as “self-cleavage”. Cleavage can also refer to the self-cleavage of two polypeptides that is induced by the addition of a non-proteolytic third peptide, as in the action of split intein system described herein.

By the term “fused” is meant covalently bonded to. For example, a first peptide is fused to a second peptide when the two peptides are covalently bonded to each other (e.g., via a peptide bond).

As used herein an “isolated” or “substantially pure” substance is one that has been separated from components which naturally accompany it. Typically, a polypeptide is substantially pure when it is at least 50% (e.g., 60%, 70%, 80%, 90%, 95%, and 99%) by weight free from the other proteins and naturally-occurring organic molecules with which it is naturally associated.

Herein, “bind” or “binds” means that one molecule recognizes and adheres to another molecule in a sample, but does not substantially recognize or adhere to other molecules in the sample. One molecule “specifically binds” another molecule if it has a binding affinity greater than about 10⁵ to 10⁶ liters/mole for the other molecule.

Nucleic acids, nucleotide sequences, proteins or amino acid sequences referred to herein can be isolated, purified, synthesized chemically, or produced through recombinant DNA technology. All of these methods are well known in the art.

As used herein, the terms “modified” or “mutated,” as in “modified intein” or “mutated intein,” refer to one or more modifications in either the nucleic acid or amino acid sequence being referred to, such as an intein, when compared to the native, or naturally occurring structure. Such modification can be a substitution, addition, or deletion. The modification can occur in one or more amino acid residues or one or more nucleotides of the structure being referred to, such as an intein.

As used herein, the term “modified peptide”, “modified protein” or “modified protein of interest” or “modified target protein” refers to a protein which has been modified.

As used herein, “operably linked” refers to the association of two or more biomolecules in a configuration relative to one another such that the normal function of the biomolecules can be performed. In relation to nucleotide sequences, “operably linked” refers to the association of two or more nucleic acid sequences, by means of enzymatic ligation or otherwise, in a configuration relative to one another such that the normal function of the sequences can be performed. For example, the nucleotide sequence encoding a pre-sequence or secretory leader is operably linked to a nucleotide sequence for a polypeptide if it is expressed as a pre-protein that participates in the secretion of the polypeptide; a promoter or enhancer is operably linked to a coding sequence if it affects the transcription of the coding sequence; and a ribosome binding site is operably linked to a coding sequence if it is positioned so as to facilitate translation of the sequence.

“Sequence homology” can refer to the situation where nucleic acid or protein sequences are similar because they have a common evolutionary origin. “Sequence homology” can indicate that sequences are very similar. Sequence similarity is observable; homology can be based on the observation. “Very similar” can mean at least 70% identity, homology or similarity; at least 75% identity, homology or similarity; at least 80% identity, homology or similarity; at least 85% identity, homology or similarity; at least 90% identity, homology or similarity; such as at least 93% or at least 95% or even at least 97% identity, homology or similarity. The nucleotide sequence similarity or homology or identity can be determined using the “Align” program of Myers et al. (1988) CABIOS 4:11-17 and available at NCBI. Additionally or alternatively, amino acid sequence similarity or identity or homology can be determined using the BlastP program (Altschul et al. Nucl. Acids Res. 25:3389-3402), and available at NCBI. Alternatively or additionally, the terms “similarity” or “identity” or “homology,” for instance, with respect to a nucleotide sequence, are intended to indicate a quantitative measure of homology between two sequences.

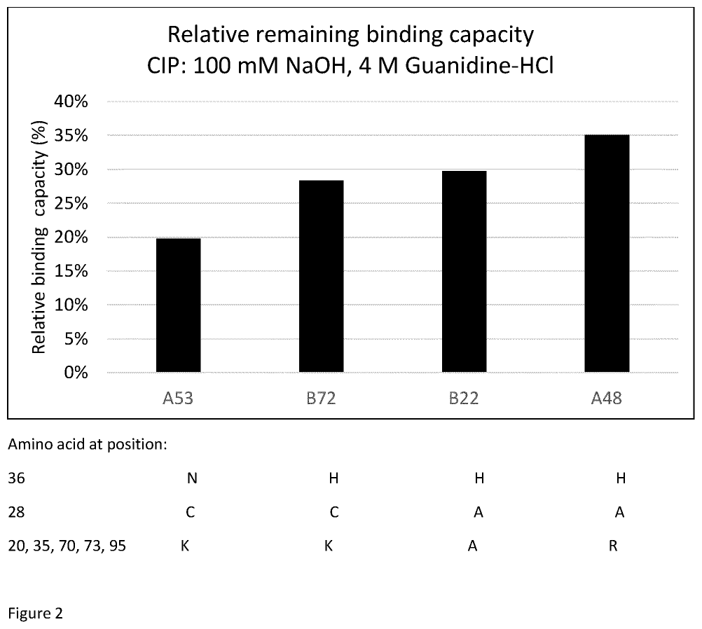

Alternatively or additionally, “similarity” with respect to sequences refers to the number of positions with identical nucleotides divided by the number of nucleotides in the shorter of the two sequences wherein alignment of the two sequences can be determined in accordance with the Wilbur and Lipman algorithm. (1983) Proc. Natl. Acad. Sci. USA 80:726. For example, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity with DNA sequences, thymidine (T) in the DNA sequence is considered equal to uracil (U) in the RNA sequence. The following references also provide algorithms for comparing the relative identity or homology or similarity of amino acid residues of two proteins, and additionally or alternatively with respect to the foregoing, the references can be used for determining percent homology or identity or similarity. Needleman et al. (1970) J. Mol. Biol. 48:444-453; Smith et al. (1983) Advances App. Math. 2:482-489; Smith et al. (1981) Nuc. Acids Res. 11:2205-2220; Feng et al. (1987) J. Molec. Evol. 25:351-360; Higgins et al. (1989) CABIOS 5:151-153; Thompson et al. (1994) Nuc. Acids Res. 22:4673-480; and Devereux et al. (1984) 12:387-395. “Stringent hybridization conditions” is a term which is well known in the art; see, for example, Sambrook, “Molecular Cloning, A Laboratory Manual” second ed., CSH Press, Cold Spring Harbor, 1989; “Nucleic Acid Hybridization, A Practical Approach”, Hames and Higgins eds., IRL Press, Oxford, 1985; see also FIG. 2 and description thereof herein wherein there is a sequence comparison.

The terms “plasmid” and “vector” and “cassette” refer to an extrachromosomal element often carrying genes which are not part of the central metabolism of the cell and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Typically, a “vector” is a modified plasmid that contains additional multiple insertion sites for cloning and an “expression cassette” that contains a DNA sequence for a selected gene product (i.e., a transgene) for expression in the host cell. This “expression cassette” typically includes a 5′ promoter region, the transgene ORF, and a 3′ terminator region, with all necessary regulatory sequences required for transcription and translation of the ORF. Thus, integration of the expression cassette into the host permits expression of the transgene ORF in the cassette.

The term “buffer” or “buffered solution” refers to solutions which resist changes in pH by the action of its conjugate acid-base range.

The term “loading buffer” or “equilibrium buffer” refers to the buffer containing the salt or salts which is mixed with the protein preparation for loading the protein preparation onto a column. This buffer is also used to equilibrate the column before loading, and to wash to column after loading the protein.

The term “wash buffer” is used herein to refer to the buffer that is passed over a column (for example) following loading of a protein of interest (such as one coupled to a C-terminal intein fragment, for example) and prior to elution of the protein of interest. The wash buffer may serve to remove one or more contaminants without substantial elution of the desired protein.

The term “elution buffer” refers to the buffer used to elute the desired protein from the column. As used herein, the term “solution” refers to either a buffered or a non-buffered solution, including water.

The term “washing” means passing an appropriate buffer through or over a solid support, such as a chromatographic resin.

The term “eluting” a molecule (e.g. a desired protein or contaminant) from a solid support means removing the molecule from such material.

The term “contaminant” or “impurity” refers to any foreign or objectionable molecule, particularly a biological macromolecule such as a DNA, an RNA, or a protein, other than the protein being purified, that is present in a sample of a protein being purified. Contaminants include, for example, other proteins from cells that express and/or secrete the protein being purified.

The term “separate” or “isolate” as used in connection with protein purification refers to the separation of a desired protein from a second protein or other contaminant or mixture of impurities in a mixture comprising both the desired protein and a second protein or other contaminant or impurity mixture, such that at least the majority of the molecules of the desired protein are removed from that portion of the mixture that comprises at least the majority of the molecules of the second protein or other contaminant or mixture of impurities.

The term “purify” or “purifying” a desired protein from a composition or solution comprising the desired protein and one or more contaminants means increasing the degree of purity of the desired protein in the composition or solution by removing (completely or partially) at least one contaminant from the composition or solution.

### N-intein Protein Variants

The invention relates to affinity chromatography and affinity tag cleavage mechanisms in a single step using a split intein system according to the invention which cleaves with broad amino acid tolerance to generate a tag less protein of interest (POI) as end product. The two halves of the intein are the affinity ligand (N-intein) and the affinity tag (C-intein) and they associate rapidly. Immobilizing one half (N-intein) on a chromatography resin enables the capture of the other half (C-intein) coupled to the POI from solution. In the presence of Zn²⁺ ions, the cleavage reaction is inhibited, enabling a stable complex to form while impurities are washed away. After impurities are eliminated, a chelator or reducing agent is added, and the cleavage reaction proceeds, enabling collection of the POI, while the intein tag remains bound non-covalently to the cognate intein linked to the chromatography resin.

Preferably the invention provides N-intein protein variant sequences of native split inteins or consensus sequences derived from native inteins and split inteins wherein, the N-intein variant is modified as compared to the native sequence or consensus sequence to eliminate all asparagine (N) amino acid residues present in the sequence. Preferably all such sequences do not include a Cysteine (C) at position 1 of the N-intein variant sequence.

Preferably, the invention provides N-intein protein variant sequences that do not include an asparagine (N) at position 36 of the variant sequence. This position is calculated according to conventional clustal alignment with native split inteins starting from the initial catalytical cysteine which is number 1. This position is conserved to N in prior art and native N-intein sequences but the present inventors have found that this position can be mutated to an amino acid that provides increased alkaline stability as compared to the native N-intein protein sequence which is important as it gives tolerance to increased pH values during for example chromatographic procedures. Preferably an amino acid that provides increased alkaline stability is histidine (H or His) or glutamine (Q or Gln).

Native intein are known in the art. A list of inteins is found in Table 1 below. All inteins have the potential to be made into split inteins while some inteins naturally exist in split form. All of the inteins found in the table either exist as split inteins or have the potential to be made into split inteins modified in accordance with the invention at position 36 such that the conserved N is replaced with another amino acid that imparts alkaline stability such as H or Q.

TABLE 1
Naturally occurring Inteins

| Intein Name | Organism Name | Organism Description |
|---|---|---|
| Eucarya | | |
| APMV Pol | Acanthomoeba polyphaga | isolate = “Rowbotham- |
| | Mimivirus | Bradford”, Virus, infects |
| | | Amoebae, taxon: 212035 |
| Abr PRP8 | Aspergillus brevipes FRR2439 | Fungi, ATCC 16899, |
| | | taxon: 75551 |
| Aca-G186AR PRP8 | Ajellomyces capsulatus G186AR | Taxon: 447093, strain |
| | | G186AR |
| Aca-H143 PRP8 | Ajellomyces capsulatus H143 | Taxon: 544712 |
| Aca-JER2004 PRP8 | Ajellomyces capsulatus (anamorph: | strain = JER2004, taxon: 5037, |
| | Histoplasma capsulatum) | Fungi |
| Aca-NAm1 PRP8 | Ajellomyces capsulatus NAm1 | strain = “NAm1”, taxon: |
| | | 339724 |
| Ade-ER3 PRP8 | Ajellomyces dermatitidis ER-3 | Human fungal |
| | | pathogen. taxon: 559297 |
| Ade-SLH14081 PRP8 | Ajellomyces dermatitidis SLH14081, | Human fungal pathogen |
| Afu-Af293 PRP8 | Aspergillus fumigatus var. | Human pathogenic fungus, |
| | ellipticus, strain Af293 | taxon: 330879 |
| Afu-FRR0163 PRP8 | Aspergillus fumigatus strain | Human pathogenic fungus, |
| | FRR0163 | taxon: 5085 |
| Afu-NRRL5109 PRP8 | Aspergillus fumigatus var. | Human pathogenic fungus, |
| | ellipticus, strain NRRL 5109 | taxon: 41121 |
| Agi-NRRL6136 PRP8 | Aspergillus giganteus Strain NRRL | Fungus, taxon: 5060 |
| | 6136 | |
| Ani-FGSCA4 PRP8 | Aspergillus nidulans FGSC A | Filamentous fungus, |
| | | taxon: 227321 |
| Avi PRP8 | Aspergillus viridinutans strain | Fungi, ATCC 16902, |
| | FRR0577 | taxon: 75553 |
| Bci PRP8 | Botrytis cinerea (teleomorph of | Plant fungal pathogen |
| | Botryotinia fuckeliana B05.10) | |
| Bde-JEL197 RPB2 | Batrachochytrium dendrobatidis | Chytrid fungus, |
| | JEL197 | isolate = “AFTOL-ID 21”, |
| | | taxon: 109871 |
| Bde-JEL423 PRP8-1 | Batrachochytrium dendrobatidis | Chytrid fungus, isolate |
| | JEL423 | JEL423, taxon 403673 |
| Bde-JEL423 PRP8-2 | Batrachochytrium dendrobatidis | Chytrid fungus, isolate |
| | JEL423 | JEL423, taxon 403673 |
| Bde-JEL423 RPC2 | Batrachochytrium dendrobatidis | Chytrid fungus, isolate |
| | JEL423 | JEL423, taxon 403673 |
| Bde-JEL423 eIF-5B | Batrachochytrium dendrobatidis | Chytrid fungus, isolate |
| | JEL423 | JEL423, taxon 403673 |
| Bfu-B05 PRP8 | Botryotinia fuckeliana B05.10 | Taxon: 332648 |
| CIV RIR1 | Chilo iridescent virus | dsDNA eucaryotic virus, |
| | | taxon: 10488 |
| CV-NY2A | Chlorella virus NY2A infects | dsDNA eucaryotic |
| ORF212392 | Chlorella NC64A, which infects | virus, taxon: 46021, Family |
| | Paramecium bursaria | Phycodnaviridae |
| CV-NY2A RIR1 | Chlorella virus NY2A infects | dsDNA eucaryotic |
| | Chlorella NC64A, which infects | virus, taxon: 46021, Family |
| | Paramecium bursaria | Phycodnaviridae |
| CZIV RIR1 | Costelytra zealandica iridescent | dsDNA eucaryotic virus, |
| | virus | Taxon: 68348 |
| Cba-WM02.98 PRP8 | Cryptococcus bacillisporus strain | Yeast, human pathogen, |
| | WM02.98 (aka Cryptococcus | taxon: 37769 |
| | neoformans gattii) | |
| Cba-WM728 PRP8 | Cryptococcus bacillisporus strain WM728 | Yeast, human pathogen, |
| | | taxon: 37769 |
| Ceu ClpP | Chlamydomonas eugametos | Green alga, taxon: 3053 |
| | (chloroplast) | |
| Cga PRP8 | Cryptococcus gattii (aka | Yeast, human pathogen |
| | Cryptococcus bacillisporus) | |
| Cgl VMA | Candida glabrata | Yeast, taxon: 5478 |
| Cla PRP8 | Cryptococcus laurentii strain | Fungi, Basidiomycete yeast, |
| | CBS139 | taxon: 5418 |
| Cmo ClpP | Chlamydomonas moewusii, strain | Green alga, chloroplast gene, |
| | UTEX 97 | taxon: 3054 |
| Cmo RPB2 (RpoBb) | Chlamydomonas moewusii, strain | Green alga, chloroplast gene, |
| | UTEX 97 | taxon: 3054 |
| Cne-A PRP8 | Filobasidiella neoformans | Yeast, human pathogen |
| (Fne-A PRP8) | (Cryptococcus neoformans) | |
| | Serotype A, PHLS 8104 | |
| Cne-AD PRP8 | Cryptococcus neoformans | Yeast, human pathogen, |
| (Fne-AD PRP8) | (Filobasidiella neoformans), | ATCC32045, taxon: 5207 |
| | Serotype AD, CBS132). | |
| Cne-JEC21 PRP8 | Cryptococcus neoformans var. | Yeast, human pathogen, |
| | neoformans JEC21 | serotype = “D” taxon: 214684 |
| Cpa ThrRS | Candida parapsilosis, strain | Yeast, Fungus, taxon: 5480 |
| | CLIB214 | |
| Cre RPB2 | Chlamydomonas reinhardtii | Green algae, taxon: 3055 |
| | (nucleus) | |
| CroV Pol | Cafeteria roenbergensis virus BV-PW1 | taxon: 693272, Giant virus |
| | | infecting marine heterotrophic |
| | | nanoflagellate |
| CroV RIR1 | Cafeteria roenbergensis virus BV-PW1 | taxon: 693272, Giant virus |
| | | infecting marine heterotrophic |
| | | nanoflagellate |
| CroV RPB2 | Cafeteria roenbergensis virus BV-PW1 | taxon: 693272, Giant virus |
| | | infecting marine heterotrophic |
| | | nanoflagellate |
| CroV Top2 | Cafeteria roenbergensis virus BV-PW1 | taxon: 693272, Giant virus |
| | | infecting marine heterotrophic |
| | | nanoflagellate |
| Cst RPB2 | Coelomomyces stegomyiae | Chytrid fungus, |
| | | isolate = “AFTOL-ID 18”, |
| | | taxon: 143960 |
| Ctr ThrRS | Candida tropicalis ATCC750 | Yeast |
| Ctr VMA | Candida tropicalis (nucleus) | Yeast |
| Ctr-MYA3404 VMA | Candida tropicalis MYA-3404 | Taxon: 294747 |
| Ddi RPC2 | Dictyostelium discoideum strain | Mycetozoa (a social amoeba) |
| | AX4 (nucleus) | |
| Dhan GLT1 | Debaryomyces hansenii CBS767 | Fungi, Anamorph: Candida |
| | | famata, taxon: 4959 |
| Dhan VMA | Debaryomyces hansenii CBS767 | Fungi, taxon: 284592 |
| Eni PRP8 | Emericella nidulans R20 | taxon: 162425 |
| | (anamorph: Aspergillus nidulans) | |
| Eni-FGSCA4 PRP8 | Emericella nidulans (anamorph: | Filamentous fungus, |
| | Aspergillus nidulans) FGSC A4 | taxon: 162425 |
| Fte RPB2 (RpoB) | Floydiella terrestris, strain UTEX | Green alga, chloroplast gene, |
| | 1709 | taxon: 51328 |
| Gth DnaB | Guillardia theta (plastid) | Cryptophyte Algae |
| HaV01 Pol | Heterosigma akashiwo virus 01 | Algal virus, taxon: 97195, |
| | | strain HaV01 |
| Hca PRP8 | Histoplasma capsulatum | Fungi, human pathogen |
| | (anamorph: Ajellomyces capsulatus) | |
| IIV6 RIR 1 | Invertebrate iridescent virus 6 | dsDNA eucaryotic virus, |
| | | taxon: 176652 |
| Kex-CBS379 VMA | Kazachstania exigua, formerly | Yeast, taxon: 34358 |
| | Saccharomyces exiguus, strain | |
| | CBS379 | |
| Kla-CBS683 VMA | Kluyveromyces lactis, strain | Yeast, taxon: 28985 |
| | CBS683 | |
| Kla-IFO1267 VMA | Kluyveromyces lactis IFO1267 | Fungi, taxon: 28985 |
| Kla-NRRLY1140 | Kluyveromyces lactis NRRL Y-1140 | Fungi, taxon: 284590 |
| VMA | | |
| Lel VMA | Lodderomyces elongisporus | Yeast |
| Mca-CBS113480 | Microsporum canis CBS 113480 | Taxon: 554155 |
| PRP8 | | |
| Nau PRP8 | Neosartorya aurata NRRL 4378 | Fungus, taxon: 41051 |
| Nfe-NRRL5534 PRP8 | Neosartorya fennelliae NRRL 5534 | Fungus, taxon: 41048 |
| Nfi PRP8 | Neosartorya fischeri | Fungi |
| Ngl-FR2163 PRP8 | Neosartorya glabra FRR2163 | Fungi, ATCC 16909, |
| | | taxon: 41049 |
| Ngl-FRR1833 PRP8 | Neosartorya glabra FRR1833 | Fungi, taxon: 41049, |
| | | (preliminary identification) |
| Nqu PRP8 | Neosartorya quadricincta, strain | |
| | NRRL 4175 | taxon: 41053 |
| Nspi PRP8 | Neosartorya spinosa FRR4595 | Fungi, taxon: 36631 |
| Pabr-Pb01 PRP8 | Paracoccidioides brasiliensis Pb01 | Taxon: 502779 |
| Pabr-Pb03 PRP8 | Paracoccidioides brasiliensis Pb03 | Taxon: 482561 |
| Pan CHS2 | Podospora anserina | Fungi, Taxon 5145 |
| Pan GLT1 | Podospora anserina | Fungi, Taxon 5145 |
| Pbl PRP8-a | Phycomyces blakesleeanus | Zygomycete fungus, strain |
| | | NRRL155 |
| Pbl PRP8-b | Phycomyces blakesleeanus | Zygomycete fungus, strain |
| | | NRRL155 |
| Pbr-Pb18 PRP8 | Paracoccidioides brasiliensis Pb18 | Fungi, taxon: 121759 |
| Pch PRP8 | Penicillium chrysogenuim | Fungus, taxon: 5076 |
| Pex PRP8 | Penicillium expansum | Fungus, taxon27334 |
| Pgu GLT1 | Pichia (Candida) guilliermondii | Fungi, Taxon 294746 |
| Pgu-alt GLT1 | Pichia (Candida) guilliermondii | Fungi |
| Pno GLT1 | Phaeosphaeria nodorum SN15 | Fungi, taxon: 321614 |
| Pno RPA2 | Phaeosphaeria nodorum SN15 | Fungi, taxon: 321614 |
| Ppu DnaB | Porphyra purpurea (chloroplast) | Red Alga |
| Pst VMA | Pichia stipitis CBS 6054, | Yeast |
| | taxon: 322104 | |
| Ptr PRP8 | Pyrenophora tritici-repentis Pt-1C-BF | Ascomycete fungus, |
| | | taxon: 426418 |
| Pvu PRP8 | Penicillium vulpinum | Fungus |
| | (formerly P. claviforme) | |
| Pye DnaB | Porphyra yezoensis chloroplast, | Red alga, |
| | cultivar U-51 | organelle = “plastid: |
| | | chloroplast”, |
| | | “taxon: 2788 |
| Sas RPB2 | Spiromyces aspiralis NRRL 22631 | Zygomycete fungus, |
| | | isolate = “AFTOL-ID |
| | | 185“, taxon: 68401 |
| Sca-CBS4309 VMA | Saccharomyces castellii, strain | Yeast, taxon: 27288 |
| | CBS4309 | |
| Sca-IFO1992 VMA | Saccharomyces castellii, strain | Yeast, taxon: 27288 |
| | IFO1992 | |
| Scar VMA | Saccharomyces cariocanus, | Yeast, taxon: 114526 |
| | strain = ″UFRJ 50791 | |
| Sce VMA | Saccharomyces cerevisiae (nucleus) | Yeast, also in Sce strains |
| | | OUT7163, OUT7045, |
| | | OUT7163, IFO1992 |
| Sce-DH1-1A VMA | Saccharomyces cerevisiae strain | Yeast, taxon: 173900, also in |
| | DH1-1A | Sce strains |
| | | OUT7900, OUT7903, |
| | | OUT7112 |
| Sce-JAY291 VMA | Saccharomyces cerevisiae JAY291 | Taxon: 574961 |
| Sce-OUT7091 VMA | Saccharomyces cerevisiae | Yeast, taxon: 4932, also in Sce |
| | OUT7091 | strains OUT7043, OUT7064 |
| Sce-OUT7112 VMA | Saccharomyces cerevisiae | Yeast, taxon: 4932, also in Sce |
| | OUT7112 | strains OUT7900, OUT7903 |
| Sce-YJM789 VMA | Saccharomyces cerevisiae strain | Yeast, taxon: 307796 |
| | YJM789 | |
| Sda VMA | Saccharomyces dairenensis, strain | Yeast, taxon: 27289, Also in |
| | CBS 421 | Sda strain IFO0211 |
| Sex-IFO1128 VMA | Saccharomyces exiguus, | Yeast, taxon: 34358 |
| | strain = “IFO1128” | |
| She RPB2 (RpoB) | Stigeoclonium helveticum, strain | Green alga, chloroplast gene, |
| | UTEX 441 | taxon: 55999 |
| Sja VMA | Schizosaccharomyces japonicus | Ascomycete fungus, |
| | yFS275 | taxon: 402676 |
| Spa VMA | Saccharomyces pastorianus | Yeast, taxon: 27292 |
| | IFO11023 | |
| Spu PRP8 | Spizellomyces punctatus | Chytrid fungus, |
| Sun VMA | Saccharomyces unisporus, strain | Yeast, taxon: 27294 |
| | CBS 398 | |
| Tgl VMA | Torulaspora globosa, strain CBS | Yeast, taxon: 48254 |
| | 764 | |
| Tpr VMA | Torulaspora pretoriensis, strain | Yeast, taxon: 35629 |
| | CBS 5080 | |
| Ure-1704 PRP8 | Uncinocarpus reesii | Filamentous fungus |
| Vpo VMA | Vanderwaltozyma polyspora, | Yeast, taxon: 36033 |
| | formerly Kluyveromyces | |
| | polysporus, strain CBS 2163 | |
| WIV RIR1 | Wiseana iridescent virus | dsDNA eucaryotic |
| | | virus, taxon: 68347 |
| Zba VMA | Zygosaccharomyces bailii, strain | Yeast, taxon: 4954 |
| | CBS 685 | |
| Zbi VMA | Zygosaccharomyces bisporus, strain | Yeast, taxon: 4957 |
| | CBS 702 | |
| Zro VMA | Zygosaccharomyces rouxii, strain | Yeast, taxon: 4956 |
| | CBS 688 | |
| Eubacteria | | |
| AP-APSE1 dpol | Acyrthosiphon pisum secondary | Bacteriophage, taxon: 67571 |
| | endosymbiot phage 1 | |
| AP-APSE2 dpol | Bacteriophage APSE-2, | Bacteriophage of Candidatus |
| | isolate = T5A | Hamiltonella defensa, |
| | | endosymbiot of |
| | | Acyrthosiphon pisum, |
| | | taxon: 340054 |
| AP-APSE4 dpol | Bacteriophage of Candidatus | Bacteriophage, taxon: 568990 |
| | Hamiltonella defensa strain 5ATac, | |
| | endosymbiot of Acyrthosiphon pisum | |
| AP-APSE5 dpol | Bacteriophage APSE-5 | Bacteriophage of Candidatus |
| | | Hamiltonella defensa, |
| | | endosymbiot of Uroleucon |
| | | rudbeckiae, taxon: 568991 |
| AP-Aaphi23 MupF | Bacteriophage Aaphi23, | Actinobacillus |
| | Haemophilus phage Aaphi23 | actinomycetemcomitans |
| | | Bacteriophage, taxon: 230158 |
| Aae RIR2 | Aquifex aeolicus strain VF5 | Thermophilic |
| | | chemolithoautotroph, |
| | | taxon: 63363 |
| Aave-AAC001 | Acidovorax avenae subsp. citrulli | taxon: 397945 |
| Aave1721 | AAC00-1 | |
| Aave-AAC001 RIR1 | Acidovorax avenae subsp. citrulli | taxon: 397945 |
| | AAC00-1 | |
| Aave-ATCC19860 | Acidovorax avenae subsp. avenae | Taxon: 643561 |
| RIR1 | ATCC 19860 | |
| Aba Hyp-02185 | Acinetobacter baumannii ACICU | taxon: 405416 |
| Ace RIR1 | Acidothermus cellulolyticus 11B | taxon: 351607 |
| Aeh DnaB-1 | Alkalilimnicola ehrlichei MLHE-1 | taxon: 187272 |
| Aeh DnaB-2 | Alkalilimnicola ehrlichei MLHE-1 | taxon: 187272 |
| Aeh RIR1 | Alkalilimnicola ehrlichei MLHE-1 | taxon: 187272 |
| AgP-S1249 MupF | Aggregatibacter phage S1249 | Taxon: 683735 |
| Aha DnaE-c | Aphanothece halophytica | Cyanobacterium, taxon: 72020 |
| Aha DnaE-n | Aphanothece halophytica | Cyanobacterium, taxon: 72020 |
| Alvi-DSM180 GyrA | Allochromatium vinosum DSM 180 | Taxon: 572477 |
| Ama MADE823 | phage uncharacterized protein | Probably prophage gene, |
| | [Alteromonas macleodii ‘Deep | taxon: 314275 |
| | ecotype’] | |
| Amax-CS328 DnaX | Arthrospira maxima CS-328 | Taxon: 513049 |
| Aov DnaE-c | Aphanizomenon ovalisporum | Cyanobacterium, taxon: 75695 |
| Aov DnaE-n | Aphanizomenon ovalisporum | Cyanobacterium, taxon: 75695 |
| Apl-C1 DnaX | Arthrospira platensis | Taxon: 118562, strain C1 |
| Arsp-FB24 DnaB | Arthrobacter species FB24 | taxon: 290399 |
| Asp DnaE-c | Anabaena species PCC7120, | Cyanobacterium, Nitrogen- |
| | (Nostoc sp. PCC7120) | fixing, taxon: 103690 |
| Asp DnaE-n | Anabaena species PCC7120, | Cyanobacterium, Nitrogen- |
| | (Nostoc sp. PCC7120) | fixing, taxon: 103690 |
| Ava DnaE-c | Anabaena variabilis ATCC29413 | Cyanobacterium, taxon: 240292 |
| Ava DnaE-n | Anabaena variabilis ATCC29413 | Cyanobacterium, taxon: 240292 |
| Avin RIR1 BIL | Azotobacter vinelandii | taxon: 354 |
| Bce-MCO3 DnaB | Burkholderia cenocepacia MC0-3 | taxon: 406425 |
| Bce-PC184 DnaB | Burkholderia cenocepacia PC184 | taxon: 350702 |
| Bse-MLS10 TerA | Bacillus selenitireducens MLS10 | Probably prophage gene, |
| | | Taxon: 439292 |
| BsuP-M1918 RIR1 | B. subtilis M1918 (prophage) | Prophage in B. subtilis M1918. |
| | | taxon: 157928 |
| BsuP-SPBc2 RIR1 | B. subtilis strain 168 Sp beta c2 | B. subtilis taxon 1423. SPbeta |
| | prophage | c2 phage, taxon: 66797 |
| Bvi IcmO | Burkholderia vietnamiensis G4 | plasmid = “pBVIE03”. |
| | | taxon: 269482 |
| CP-P1201 Thy1 | Corynebacterium phage P1201 | lytic bacteriophage P1201 |
| | | from Corynebacterium |
| | | glutamicum NCHU |
| | | 87078. Viruses; dsDNA |
| | | viruses, taxon: 384848 |
| Cag RIR1 | Chlorochromatium aggregatum | Motile, phototrophic consortia |
| | | Anoxygenic |
| Cau SpoVR | Chloroflexus aurantiacus J-10-fl | phototroph, taxon: 324602 |
| CbP-C-St RNR | Clostridium botulinum phage C-St | Phage, specific_host = |
| | | “Clostridium |
| | | botulinum type C strain |
| | | C-Stockholm, taxon: 12336 |
| CbP-D1873 RNR | Clostridium botulinum phage D | Ssp. phage from Clostridium |
| | | botulinum type D strain, 1873, |
| | | taxon: 29342 |
| Cbu-Dugway DnaB | Coxiella burnetii Dugway 5J108-111 | Proteobacteria; Legionellales; |
| | | taxon: 434922 |
| Cbu-Goat DnaB | Coxiella burnetii ‘MSU Goat Q177’ | Proteobacteria; Legionellales; |
| | | taxon: 360116 |
| Cbu-RSA334 DnaB | Coxiella burnetii RSA 334 | Proteobacteria; Legionellales; |
| | | taxon: 360117 |
| Cbu-RSA493 DnaB | Coxiella burnetii RSA 493 | Proteobacteria; Legionellales; |
| | | taxon: 227377 |
| Cce Hyp1-Csp-2 | Cyanothece sp. ATCC 51142 | Marine unicellular |
| | | diazotrophic cyanobacterium, |
| | | taxon: 43989 |
| Cch RIR1 | Chlorobium chlorochromatii CaD3 | taxon: 340177 |
| Ccy Hyp1-Csp-1 | Cyanothece sp. CCY0110 | Cyanobacterium, |
| | | taxon: 391612 |
| Ccy Hyp1-Csp-2 | Cyanothece sp. CCY0110 | Cyanobacterium, |
| | | taxon: 391612 |
| Cfl-DSM20109 DnaB | Cellulomonas flavigena DSM 20109 | Taxon: 446466 |
| Chy RIR1 | Carboxydothermus | Thermophile, taxon = 246194 |
| | hydrogenoformans Z-2901 | |
| Ckl PTerm | Clostridium kluyveri DSM 555 | plasmid = “pCKL555A”, |
| | | taxon: 431943 |
| Cra-CS505 DnaE-c | Cylindrospermopsis raciborskii CS-505 | Taxon: 533240 |
| Cra-CS505 DnaE-n | Cylindrospermopsis raciborskii CS-505 | Taxon: 533240 |
| Cra-CS505 GyrB | Cylindrospermopsis raciborskii CS-505 | Taxon: 533240 |
| Csp-CCY0110 | Cyanothece sp. CCY0110 | Taxon: 391612 |
| DnaE-C | | |
| Csp-CCY0110 | Cyanothece sp. CCY0110 | Taxon: 391612 |
| DnaE-n | | |
| Csp-PCC7424 | Cyanothece sp. PCC 7424 | Cyanobacterium, taxon: 65393 |
| DnaE-c | | |
| Csp-PCC7424 | Cyanothece sp. PCC7424 | Cyanobacterium, taxon: 65393 |
| DnaE-n | | |
| Csp-PCC7425 DnaB | Cyanothece sp. PCC 7425 | Taxon: 395961 |
| Csp-PCC7822 | Cyanothece sp. PCC 7822 | Taxon: 497965 |
| DnaE-n | | |
| Csp-PCC8801 | Cyanothece sp. PCC 8801 | Taxon: 41431 |
| DnaE-C | | |
| Csp-PCC8801 | Cyanothece sp. PCC 8801 | Taxon: 41431 |
| DnaE-n | | |
| Cth ATPase BIL | Clostridium thermocellum | ATCC27405, taxon: 203119 |
| Cth-ATCC27405 | Clostridium thermocellum | Probable prophage, |
| TerA | ATCC27405 | ATCC27405, taxon: 203119 |
| Cth-DSM2360 TerA | Clostridium thermocellum DSM | Probably prophage |
| | 2360 | gene, Taxon: 572545 |
| Cwa DnaB | Crocosphaera watsonii WH 8501 | taxon: 165597 |
| | (Synechocystis sp. WH 8501) | |
| Cwa DnaE-c | Crocosphaera watsonii WH 8501 | Cyanobacterium, |
| | (Synechocystis sp. WH 8501) | taxon: 165597 |
| Cwa DnaE-n | Crocosphaera watsonii WH 8501 | Cyanobacterium, |
| | (Synechocystis sp. WH 8501) | taxon: 165597 |
| Cwa PEP | Crocosphaera watsonii WH 8501 | taxon: 165597 |
| | (Synechocystis sp. WH 8501) | |
| Cwa RIR1 | Crocosphaera watsonii WH 8501 | taxon: 165597 |
| | (Synechocystis sp. WH 8501) | |
| Daud RIR1 | Candidatus Desulforudis | taxon: 477974 |
| | audaxviator MP104C | |
| Dge DnaB | Deinococcus geothermalis | Thermophilic, radiation |
| | DSM11300 | resistant |
| Dha-DCB2 RIR1 | Desulfitobacterium hafniense DCB-2 | Anaerobic dehalogenating |
| | | bacteria, taxon: 49338 |
| Dha-Y51 RIR1 | Desulfitobacterium hafniense Y51 | Anaerobic dehalogenating |
| | | bacteria, taxon: 138119 |
| Dpr-MLMSI RIR1 | delta proteobacterium MLMS-1 | Taxon: 262489 |
| Dra RIR1 | Deinococcus radiodurans R1, TIGR | Radiation resistant, |
| | strain | taxon: 1299 |
| Dra Snf2-c | Deinococcus radiodurans R1, TIGR | Radiation and DNA damage |
| | strain | resistent, taxon: 1299 |
| Dra Snf2-n | Deinococcus radiodurans R1, TIGR | Radiation and DNA damage |
| | strain | resistent, taxon: 1299 |
| Dra-ATCC13939 | Deinococcus radiodurans R1, | Radiation and DNA damage |
| Snf2 | ATCC13939/Brooks & Murray | resistent, taxon: 1299 |
| | strain | |
| Dth UDP GD | Dictyoglomus thermophilum H-6-12 | strain = “H-6-12; ATCC 35947, |
| | | taxon: 309799 |
| Dvul ParB | Desulfovibrio vulgaris subsp. | taxon: 391774 |
| | vulgaris DP4 | |
| EP-Min27 Primase | Enterobacteria phage Min27 | bacteriphage of |
| | | host = “Escherichia coli |
| | | 0157: H7 str. Min27” |
| Fal DnaB | Frankia alni ACN14a | Plant symbiot, taxon: 326424 |
| Fsp-CcI3 RIR1 | Frankia species CcI3 | taxon: 106370 |
| Gob DnaE | Gemmata obscuriglobus UQM2246 | Taxon 114, TIGR genome |
| | | strain, budding bacteria |
| Gob Hyp | Gemmata obscuriglobus UQM2246 | Taxon 114, TIGR genome |
| | | strain, budding bacteria |
| Gvi DnaB | Gloeobacter violaceus, PCC 7421 | taxon: 33072 |
| Gvi RIR1-1 | Gloeobacter violaceus, PCC 7421 | taxon: 33072 |
| Gvi RIR1-2 | Gloeobacter violaceus, PCC 7421 | taxon: 33072 |
| Hhal DnaB | Halorhodospira halophila SL1 | taxon: 349124 |
| Kfl-DSM17836 DnaB | Kribbella flavida DSM 17836 | Taxon: 479435 |
| Kra DnaB | Kineococcus radiotolerans | Radiation resistant |
| | SRS30216 | |
| LLP-KSY1 PolA | Lactococcus phage KSY1 | Bacteriophage, taxon: 388452 |
| LP-phiHSIC Helicase | Listonella pelagia phage phiHSIC | taxon: 310539, a |
| | | pseudotemperate marine |
| | | phage of Listonella pelagia |
| Lsp-PCC8106 GyrB | Lyngbya sp. PCC 8106 | Taxon: 313612 |
| MP-Be DnaB | Mycobacteriophage Bethlehem | Bacteriophage, taxon: 260121 |
| MP-Be gp51 | Mycobacteriophage Bethlehem | Bacteriophage, taxon: 260121 |
| MP-Catera gp206 | Mycobacteriophage Catera | Mycobacteriophage, |
| | | taxon: 373404 |
| MP-KBG gp53 | Mycobacterium phage KBG | Taxon: 540066 |
| MP-Mcjw1 DnaB | Mycobacteriophage CJW1 | Bacteriophage, taxon: 205869 |
| MP-Omega DnaB | Mycobacteriophage Omega | Bacteriophage, taxon: 205879 |
| MP-U2 gp50 | Mycobacteriophage U2 | Bacteriophage, taxon: 260120 |
| Maer-NIES843 DnaB | Microcystis aeruginosa NIES-843 | Bloom-forming toxic |
| | | cyanobacterium, taxon: 449447 |
| Maer-NIES843 | Microcystis aeruginosa NIES-843 | Bloom-forming toxic |
| DnaE-C | | cyanobacterium, taxon: 449447 |
| Maer-NIES843 | Microcystis aeruginosa NIES-843 | Bloom-forming toxic |
| DnaE-n | | cyanobacterium, taxon: 449447 |
| Mau-ATCC27029 | Micromonospora aurantiaca ATCC | Taxon: 644283 |
| GyrA | 27029 | |
| Mav-104 DnaB | Mycobacterium avium 104 | taxon: 243243 |
| Mav-ATCC25291 | Mycobacterium avium subsp. avium | Taxon: 553481 |
| DnaB | ATCC 25291 | |
| Mav-ATCC35712 | Mycobacterium avium | ATCC35712, taxon 1764 |
| DnaB | | |
| Mav-PT DnaB | Mycobacterium avium subsp. | taxon: 262316 |
| | paratuberculosis str. k10 | |
| Mbo Pps1 | Mycobacterium bovis subsp. bovis | strain = “AF2122/97”, |
| | AF2122/97 | taxon: 233413 |
| Mbo RecA | Mycobacterium bovis subsp. bovis | taxon: 233413 |
| | AF2122/97 | |
| Mbo SufB | Mycobacterium bovis subsp. bovis | taxon: 233413 |
| (Mbo Pps1) | AF2122/97 | |
| Mbo-1173P DnaB | Mycobacterium bovis BCG Pasteur | strain = BCG Pasteur |
| | 1173P | 1173P2,, taxon: 410289 |
| Mbo-AF2122 DnaB | Mycobacterium bovis subsp. bovis | strain = “AF2122/97”, |
| | AF2122/97 | taxon: 233413 |
| Mca MupF | Methylococcus capsulatus Bath, | prophage MuMc02, |
| | prophage MuMc02 | taxon: 243233 |
| Mca RIR1 | Methylococcus capsulatus Bath | taxon: 243233 |
| Mch RecA | Mycobacterium chitae | IP14116003, taxon: 1792 |
| Mcht-PCC7420 | Microcoleus chthonoplastes | Cyanobacterium, |
| DnaE-1 | PCC7420 | taxon: 118168 |
| Mcht-PCC7420 | Microcoleus chthonoplastes | Cyanobacterium, |
| DnaE-2c | PCC7420 | taxon: 118168 |
| Mcht-PCC7420 | Microcoleus chthonoplastes | Cyanobacterium, |
| DnaE-2n | PCC7420 | taxon: 118168 |
| Mcht-PCC7420 GyrB | Microcoleus chthonoplastes PCC 7420 | Taxon: 118168 |
| Mcht-PCC7420 | Microcoleus chthonoplastes PCC | Taxon: 118168 |
| RIR1-1 | 7420 | |
| Mcht-PCC7420 | Microcoleus chthonoplastes PCC | Taxon: 118168 |
| RIR1-2 | 7420 | |
| Mex Helicase | Methylobacterium extorquens AMI | Alphaproteobacteria |
| Mex TrbC | Methylobacterium extorquens AMI | Alphaproteobacteria |
| Mfa RecA | Mycobacterium fallax | CITP8139, taxon: 1793 |
| Mfl GyrA | Mycobacterium flavescens Fla0 | taxon: 1776, reference |
| | | #930991 |
| Mfl RecA | Mycobacterium flavescens Fla0 | strain = Fla0, taxon: 1776, ref. |
| | | #930991 |
| Mfl-ATCC14474 | Mycobacterium flavescens, | strain = ATCC14474, taxon: |
| RecA | ATCC14474 | 1776, ref #930991 |
| Mfl-PYR-GCK DnaB | Mycobacterium flavescens PYR-GCK | taxon: 350054 |
| Mga GyrA | Mycobacterium gastri | HP4389, taxon: 1777 |
| Mga RecA | Mycobacterium gastri | HP4389, taxon: 1777 |
| Mga SufB | Mycobacterium gastri | HP4389, taxon: 1777 |
| (Mga Pps1) | | |
| Mgi-PYR-GCK DnaB | Mycobacterium gilvum PYR-GCK | taxon: 350054 |
| Mgi-PYR-GCK GyrA | Mycobacterium gilvum PYR-GCK | taxon: 350054 |
| Mgo GyrA | Mycobacterium gordonae | taxon: 1778, reference number |
| | | 930835 |
| Min-1442 DnaB | Mycobacterium intracellulare | strain 1442, taxon: 1767 |
| Min-ATCC13950 | Mycobacterium intracellulare | Taxon: 487521 |
| GyrA | ATCC 13950 | |
| Mkas GyrA | Mycobacterium kansasii | taxon: 1768 |
| Mkas-ATCC12478 | Mycobacterium kansasii ATCC 12478 | Taxon: 557599 |
| GyrA | | |
| Mle-Br4923 GyrA | Mycobacterium leprae Br4923 | Taxon: 561304 |
| Mle-TN DnaB | Mycobacterium leprae, strain TN | Human pathogen, taxon: 1769 |
| Mle-TN GyrA | Mycobacterium leprae TN | Human pathogen, |
| | | STRAIN = TN, taxon: 1769 |
| Mle-TN RecA | Mycobacterium leprae, strain TN | Human pathogen, taxon: 1769 |
| Mle-TN SufB | Mycobacterium leprae | Human pathogen, taxon: 1769 |
| (Mle Pps1) | | |
| Mma GyrA | Mycobacterium malmoense | taxon: 1780 |
| Mmag Magn8951 | Magnetospirillum magnetotacticum | Gram negative, taxon: 272627 |
| BIL | MS-1 | |
| Msh RecA | Mycobacterium shimodei | ATCC27962, taxon: 29313 |
| Msm DnaB-1 | Mycobacterium smegmatis MC2 | MC2 155, taxon: 246196 |
| | 155 | |
| Msm DnaB-2 | Mycobacterium smegmatis MC2 | MC2 155, taxon: 246196 |
| | 155 | |
| Msp-KMS DnaB | Mycobacterium species KMS | taxon: 189918 |
| Msp-KMS GyrA | Mycobacterium species KMS | taxon: 189918 |
| Msp-MCS DnaB | Mycobacterium species MCS | taxon: 164756 |
| Msp-MCS GyrA | Mycobacterium species MCS | taxon: 164756 |
| Mthe RecA | Mycobacterium thermoresistibile | ATCC19527, taxon: 1797 |
| Mtu SufB (Mtu Pps1) | Mycobacterium tuberculosis strains | Human pathogen, taxon: 83332 |
| | H37Rv & CDC1551 | |
| Mtu-C RecA | Mycobacterium tuberculosis C | Taxon: 348776 |
| Mtu-CDC1551 DnaB | Mycobacterium tuberculosis, | Human pathogen, taxon: 83332 |
| | CDC1551 | |
| Mtu-CPHL RecA | Mycobacterium tuberculosis | Taxon: 611303 |
| | CPHL_A | |
| Mtu-Canetti RecA | Mycobacterium tuberculosis/ | Taxon: 1773 |
| | strain = “Canetti” | |
| Mtu-EAS054 RecA | Mycobacterium tuberculosis | Taxon: 520140 |
| | EAS054 | |
| Mtu-F11 DnaB | Mycobacterium tuberculosis, strain | taxon: 336982 |
| | F11 | |
| Mtu-H37Ra DnaB | Mycobacterium tuberculosis H37Ra | ATCC 25177, taxon: 419947 |
| Mtu-H37Rv DnaB | Mycobacterium tuberculosis H37Rv | Human pathogen, taxon: 83332 |
| Mtu-H37Rv RecA | Mycobacterium tuberculosis | Human pathogen, taxon: 83332 |
| | H37Rv, Also CDC1551 | |
| Mtu-Haarlem DnaB | Mycobacterium tuberculosis str. | Taxon: 395095 |
| | Haarlem | |
| Mtu-K85 RecA | Mycobacterium tuberculosis K85 | Taxon: 611304 |
| Mtu-R604 RecA-n | Mycobacterium tuberculosis | Taxon: 555461 |
| | ‘98-R604 INH-RIF-EM’ | |
| Mtu-So93 RecA | Mycobacterium tuberculosis | Human pathogen, taxon: 1773 |
| | So93/sub_species = “Canetti” | |
| Mtu-T17 RecA-c | Mycobacterium tuberculosis T17 | Taxon: 537210 |
| Mtu-T17 RecA-n | Mycobacterium tuberculosis T17 | Taxon: 537210 |
| Mtu-T46 RecA | Mycobacterium tuberculosis T46 | Taxon: 611302 |
| Mtu-T85 RecA | Mycobacterium tuberculosis T85 | Taxon: 520141 |
| Mtu-T92 RecA | Mycobacterium tuberculosis T92 | Taxon: 515617 |
| Mvan DnaB | Mycobacterium vanbaalenii PYR-1 | taxon: 350058 |
| Mvan GyrA | Mycobacterium vanbaalenii PYR-1 | taxon: 350058 |
| Mxa RAD25 | Myxococcus xanthus DK1622 | Deltaproteobacteria |
| Mxe GyrA | Mycobacterium xenopi strain | taxon: 1789 |
| | IMM5024 | |
| Naz-0708 RIR1-1 | Nostoc azollae 0708 | Taxon: 551115 |
| Naz-0708 RIR1-2 | Nostoc azollae 0708 | Taxon: 551115 |
| Nfa DnaB | Nocardia farcinica IFM 10152 | taxon: 247156 |
| Nfa Nfa 15250 | Nocardia farcinica IFM 10152 | taxon: 247156 |
| Nfa RIR1 | Nocardia farcinica IFM 10152 | taxon: 247156 |
| Nosp-CCY9414 | Nodularia spumigena CCY9414 | Taxon: 313624 |
| DnaE-n | | |
| Npu DnaB | Nostoc punctiforme | Cyanobacterium, taxon: 63737 |
| Npu GyrB | Nostoc punctiforme | Cyanobacterium, taxon: 63737 |
| Npu-PCC73102 | Nostoc punctiforme PCC73102 | Cyanobacterium, taxon: 63737, |
| DnaE-c | | ATCC29133 |
| Npu-PCC73102 | Nostoc punctiforme PCC73102 | Cyanobacterium, taxon: 63737, |
| DnaE-n | | ATCC29133 |
| Nsp-JS614 DnaB | Nocardioides species JS614 | taxon: 196162 |
| Nsp-JS614 TOPRIM | Nocardioides species JS614 | taxon: 196162 |
| Nsp-PCC7120 DnaB | Nostoc species PCC7120, | Cyanobacterium, Nitrogen- |
| | (Anabaena sp. PCC7120) | fixing, taxon: 103690 |
| Nsp-PCC7120 | Nostoc species PCC7120, | Cyanobacterium, Nitrogen- |
| DnaE-c | (Anabaena sp. PCC7120) | fixing, taxon: 103690 |
| Nsp-PCC7120 | Nostoc species PCC7120, | Cyanobacterium, Nitrogen- |
| DnaE-n | (Anabaena sp. PCC7120) | fixing, taxon: 103690 |
| Nsp-PCC7120 RIR1 | Nostoc species PCC7120, | Cyanobacterium, Nitrogen- |
| | (Anabaena sp. PCC7120) | fixing, taxon: 103690 |
| Oli DnaE-c | Oscillatoria limnetica str. ‘Solar Lake’ | Cyanobacterium, taxon: 262926 |
| Oli DnaE-n | Oscillatoria limnetica str. ‘Solar Lake’ | Cyanobacterium, taxon: 262926 |
| PP-PhiEL Helicase | Pseudomonas aeruginosa phage | Phage infects Pseudomonas |
| | phiEL | aeruginosa, taxon: 273133 |
| PP-PhiEL ORF11 | Pseudomonas aeruginosa phage | phage infects Pseudomonas |
| | phiEL | aeruginosa, taxon: 273133 |
| PP-PhiEL ORF39 | Pseudomonas aeruginosa phage | Phage infects Pseudomonas |
| | phiEL | aeruginosa, taxon: 273133 |
| PP-PhiEL ORF40 | Pseudomonas aeruginosa phage | phage infects Pseudomonas |
| | phiEL | aeruginosa, taxon: 273133 |
| Pfl Fha BIL | Pseudomonas fluorescens Pf-5 | Plant commensal organism, |
| | | taxon: 220664 |
| Plut RIR1 | Pelodictyon luteolum DSM 273 | Green sulfur bacteria, Taxon |
| | | 319225 |
| Pma-EXH1 GyrA | Persephonella marina EX-H1 | Taxon: 123214 |
| Pma-ExH1 DnaE | Persephonella marina EX-H1 | Taxon: 123214 |
| Pna RIR1 | Polaromonas naphthalenivorans | taxon: 365044 |
| | CJ2 | |
| Pnuc DnaB | Polynucleobacter sp. | taxon: 312153 |
| | QLW-P1DMWA-1 | |
| Posp-JS666 DnaB | Polaromonas species JS666 | taxon: 296591 |
| Posp-JS666 RIR1 | Polaromonas species JS666 | taxon: 296591 |
| Pssp-A1-1 Fha | Pseudomonas species A1-1 | |
| Psy Fha | Pseudomonas syringae pv. tomato | Plant (tomato) pathogen, |
| | str. DC3000 | taxon: 223283 |
| Rbr-D9 GyrB | Raphidiopsis brookii D9 | Taxon: 533247 |
| Rce RIR1 | Rhodospirillum centenum SW | taxon: 414684, ATCC 51521 |
| Rer-SK121 DnaB | Rhodococcus erythropolis SK121 | Taxon: 596309 |
| Rma DnaB | Rhodothermus marinus | Thermophile, taxon: 29549 |
| Rma-DSM4252 DnaB | Rhodothermus marinus DSM 4252 | Taxon: 518766 |
| Rma-DSM4252 DnaE | Rhodothermus marinus DSM 4252 | Thermophile, taxon: 518766 |
| Rsp RIR1 | Roseovarius species 217 | taxon: 314264 |
| SaP-SETP12 dpol | Salmonella phage SETP12 | Phage, taxon: 424946 |
| SaP-SETP3 Helicase | Salmonella phage SETP3 | Phage, taxon: 424944 |
| SaP-SETP3 dpol | Salmonella phage SETP3 | Phage, taxon: 424944 |
| SaP-SETP5 dpol | Salmonella phage SETP5 | Phage, taxon: 424945 |
| Sare DnaB | Salinispora arenicola CNS-205 | taxon: 391037 |
| Sav RecG Helicase | Streptomyces avermitilis MA-4680 | taxon: 227882, ATCC 31267 |
| Sel-PC6301 RIR1 | Synechococcus elongatus PCC 6301 | taxon: 269084 Berkely strain |
| | | 6301~equivalent name: Ssp |
| | | PCC6301~synonym: |
| | | Anacystis nudulans |
| Sel-PC7942 DnaE-c | Synechococcus elongatus PC7942 | taxon: 1140 |
| Sel-PC7942 DnaE-n | Synechococcus elongatus PC7942 | taxon: 1140 |
| Sel-PC7942 RIR1 | Synechococcus elongatus PC7942 | taxon: 1140 |
| Sel-PCC6301 DnaE-c | Synechococcus elongatus PCC6301 | Cyanobacterium, |
| | and PCC7942 | taxon: 269084, “Berkely strain |
| | | 6301~equivalent name: |
| | | Synechococcus sp. PCC |
| | | 6301~synonym: Anacystis |
| | | nudulans” |
| Sel-PCC6301 DnaE-n | Synechococcus elongatus PCC6301 | Cyanobacterium, |
| | | taxon: 269084 “Berkely strain |
| | | 6301~equivalent name: |
| | | Synechococcus sp. PCC |
| | | 6301~synonym: Anacystis |
| | | nudulans” |
| Sep RIR1 | Staphylococcus epidermidis RP62A | taxon: 176279 |
| ShP-Sfv-2a-2457T-n | Shigella flexneri 2a str. 2457T | Putative bacteriphage |
| Primase | | |
| ShP-Sfv-2a-301-n | Shigella flexneri 2a str. 301 | Putative bacteriphage |
| Primase | | |
| ShP-Sfv-5 Primase | Shigella flexneri 5 str. 8401 | Bacteriphage, isolation_source_ |
| | | epidemic, taxon: 373384 |
| SoP-SO1 dpol | Sodalis phage SO-1 | Phage/isolation_source = |
| | | “Sodalis glossinidius |
| | | strain GA-SG, |
| | | secondary symbiont of |
| | | Glossina austeni (Newstead)” |
| Spl DnaX | Spirulina platensis, strain C1 | Cyanobacterium, taxon: 1156 |
| Sru DnaB | Salinibacter ruber DSM 13855 | taxon: 309807, strain = “DSM |
| | | 13855; M31” |
| Sru PolBc | Salinibacter ruber DSM 13855 | taxon: 309807, strain = “DSM |
| | | 13855; M31” |
| Sru RIR1 | Salinibacter ruber DSM 13855 | taxon: 309807, strain = “DSM |
| | | 13855; M31” |
| Ssp DnaB | Synechocystis species, strain | Cyanobacterium, taxon: 1148 |
| | PCC6803 | |
| Ssp DnaE-c | Synechocystis species, strain | Cyanobacterium, taxon: 1148 |
| | PCC6803 | |
| Ssp DnaE-n | Synechocystis species, strain | Cyanobacterium, taxon: 1148 |
| | PCC6803 | |
| Ssp DnaX | Synechocystis species, strain | Cyanobacterium, taxon: 1148 |
| | PCC6803 | |
| Ssp GyrB | Synechocystis species, strain | Cyanobacterium, taxon: 1148 |
| | PCC6803 | |
| Ssp-JA2 DnaB | Synechococcus species JA-2- | Cyanobacterium, Taxon: |
| | 3B′a(2-13) | 321332 |
| Ssp-JA2 RIR1 | Synechococcus species JA-2- | Cyanobacterium, Taxon: |
| | 3B′a(2-13) | 321332 |
| Ssp-JA3 DnaB | Synechococcus species JA-3-3Ab | Cyanobacterium, Taxon: |
| | | 321327 |
| Ssp-JA3 RIR1 | Synechococcus species JA-3-3 Ab | Cyanobacterium, Taxon: |
| | | 321327 |
| Ssp-PCC7002 DnaE-c | Synechocystis species, strain PCC | Cyanobacterium, taxon: 32049 |
| Ssp-PCC7002 DnaE-n | Synechocystis species, strain PCC 7002 | Cyanobacterium, taxon: 32049 |
| Ssp-PCC7335 RIR1 | Synechococcus sp. PCC 7335 | Taxon: 91464 |
| StP-Twort ORF6 | Staphylococcus phage Twort | Phage, taxon 55510 |
| Susp-NBC371 DnaB | Sulfurovum sp. NBC37-1 | taxon: 387093 |
| Intein | | |
| Taq-Y51MC23 DnaE | Thermus aquaticus Y51MC23 | Taxon: 498848 |
| Taq-Y51MC23 RIR1 | Thermus aquaticus Y51MC23 | Taxon: 498848 |
| Tcu-DSM43183 | Thermomonospora curvata DSM | Taxon: 471852 |
| RecA | 43183 | |
| Tel DnaE-c | Thermosynechococcus elongatus | Cyanobacterium, taxon: 197221 |
| | BP-1 | |
| Tel DnaE-n | Thermosynechococcus elongatus | Cyanobacterium, |
| | BP-1 | |
| Ter DnaB-1 | Trichodesmium erythraeum | Cyanobacterium, taxon: 203124 |
| | IMS101 | |
| Ter DnaB-2 | Trichodesmium erythraeum | Cyanobacterium, taxon: 203124 |
| | IMS101 | |
| Ter DnaE-1 | Trichodesmium erythraeum | Cyanobacterium, taxon: 203124 |
| | IMS101 | |
| Ter DnaE-2 | Trichodesmium erythraeum | Cyanobacterium, taxon: 203124 |
| | IMS101 | |
| Ter DnaE-3c | Trichodesmium erythraeum | Cyanobacterium, taxon: 203124 |
| | IMS101 | |
| Ter DnaE-3n | Trichodesmium erythraeum | Cyanobacterium, taxon: 203124 |
| | IMS101 | |
| Ter GyrB | Trichodesmium erythraeum | Cyanobacterium, taxon: 203124 |
| | IMS101 | |
| Ter Ndse-1 | Trichodesmium erythraeum | Cyanobacterium, taxon: 203124 |
| | IMS101 | |
| Ter Ndse-2 | Trichodesmium erythraeum | Cyanobacterium, taxon: 203124 |
| | IMS101 | |
| Ter RIR1-1 | Trichodesmium erythraeum | Cyanobacterium, taxon: 203124 |
| | IMS101 | |
| Ter RIR1-2 | Trichodesmium erythraeum | Cyanobacterium, taxon: 203124 |
| | IMS101 | |
| Ter RIR1-3 | Trichodesmium erythraeum | Cyanobacterium, taxon: 203124 |
| | IMS101 | |
| Ter RIR1-4 | Trichodesmium erythraeum | Cyanobacterium, taxon: 203124 |
| | IMS101 | |
| Ter Snf2 | Trichodesmium erythraeum | Cyanobacterium, taxon: 203124 |
| | IMS101 | |
| Ter Thy X | Trichodesmium erythraeum | Cyanobacterium, taxon: 203124 |
| | IMS101 | |
| Tfus RecA-1 | Thermobifida fusca YX | Thermophile, taxon: 269800 |
| Tfus RecA-2 | Thermobifida fusca YX | Thermophile, taxon: 269800 |
| Tfus Tfu2914 | Thermobifida fusca YX | Thermophile, taxon: 269800 |
| Thsp-K90 RIR1 | Thioalkalivibrio sp. K90mix | Taxon: 396595 |
| Tth-DSM571 RIR1 | Thermoanaerobacterium | Taxon: 580327 |
| | thermosaccharolyticum DSM 571 | |
| Tth-HB27 DnaE-1 | Thermus thermophilus HB27 | thermophile, taxon: 262724 |
| Tth-HB27 DnaE-2 | Thermus thermophilus HB27 | thermophile, taxon: 262724 |
| Tth-HB27 RIR1-1 | Thermus thermophilus HB27 | thermophile, taxon: 262724 |
| Tth-HB27 RIR1-2 | Thermus thermophilus HB27 | thermophile, taxon: 262724 |
| Tth-HB8 DnaE-1 | Thermus thermophilus HB8 | thermophile, taxon: 300852 |
| Tth-HB8 DnaE-2 | Thermus thermophilus HB8 | thermophile, taxon: 300852 |
| Tth-HB8 RIR1-1 | Thermus thermophilus HB8 | thermophile, taxon: 300852 |
| Tth-HB8 RIR1-2 | Thermus thermophilus HB8 | thermophile, taxon: 300852 |
| Tvu DnaE-c | Thermosynechococcus vulcanus | Cyanobacterium, taxon: 32053 |
| Tvu DnaE-n | Thermosynechococcus vulcanus | Cyanobacterium, taxon: 32053 |
| Tye RNR-1 | Thermodesulfovibrio yellowstonii | taxon: 289376 |
| | DSM 11347 | |
| Tye RNR-2 | Thermodesulfovibrio yellowstonii | taxon: 289376 |
| | DSM 11347 | |
| Archaea | | |
| Ape APE0745 | Aeropyrum pernix K1 | Thermophile, taxon: 56636 |
| Cme-boo Pol-II | Candidatus Methanoregula boonei | taxon: 456442 |
| | 6A8 | |
| Fac-Fer1 RIR1 | Ferroplasma acidarmanus, | strain Fer1, eats iron |
| | taxon: 97393 and taxon 261390 | |
| Fac-Ferl SufB | Ferroplasma acidarmanus | strain fer1, eats |
| (Fac Pps1) | | iron, taxon: 97393 |
| Fac-TypeI RIR1 | Ferroplasma acidarmanus type I, | Eats iron, taxon 261390 |
| Fac-typeI SufB | Ferroplasma acidarmanus | Eats iron, taxon: 261390 |
| (Fac Pps1) | | |
| Hma CDC21 | Haloarcula marismortui ATCC | taxon: 272569, |
| | 43049 | |
| Hma Pol-II | Haloarcula marismortui ATCC | taxon: 272569, |
| | 43049 | |
| Hma PolB | Haloarcula marismortui ATCC | taxon: 272569, |
| | 43049 | |
| Hma TopA | Haloarcula marismortui ATCC | taxon: 272569 |
| | 43049 | |
| Hmu-DSM12286 | Halomicrobium mukohataei DSM | taxon: 485914 (Halobacteria) |
| MCM | 12286 | |
| Hmu-DSM12286 | Halomicrobium mukohataei DSM | Taxon: 485914 |
| PolB | 12286 | |
| Hsa-R1 MCM | Halobacterium salinarum R-1 | Halophile, |
| | | taxon: 478009, strain = “R1; |
| | | DSM 671” |
| Hsp-NRC1 CDC21 | Halobacterium species NRC-1 | Halophile, taxon: 64091 |
| Hsp-NRC1 Pol-II | Halobacterium salinarum NRC-1 | Halophile, taxon: 64091 |
| Hut MCM-2 | Halorhabdus utahensis DSM 12940 | taxon: 519442 |
| Hut-DSM12940 | Halorhabdus utahensis DSM 12940 | taxon: 519442 |
| MCM-1 | | |
| Hvo PolB | Haloferax volcanii DS70 | taxon: 2246 |
| Hwa GyrB | Haloquadratum walsbyi DSM | Halophile, taxon: 362976, |
| | 16790 | strain: DSM 16790 = |
| | | HBSQ001 |
| Hwa MCM-1 | Haloquadratum walsbyi DSM | Halophile, taxon: 362976, |
| | 16790 | strain: DSM 16790 = |
| | | HBSQ001 |
| Hwa MCM-2 | Haloquadratum walsbyi DSM | Halophile, taxon: 362976, |
| | 16790 | strain: DSM 16790 = |
| | | HBSQ001 |
| Hwa MCM-3 | Haloquadratum walsbyi DSM | Halophile, taxon: 362976, |
| | 16790 | strain: DSM 16790 = |
| | | HBSQ001 |
| Hwa MCM-4 | Haloquadratum walsbyi DSM | Halophile, taxon: 362976, |
| | 16790 | strain: DSM 16790 = |
| | | HBSQ001 |
| Hwa Pol-II-1 | Haloquadratum walsbyi DSM | Halophile, taxon: 362976, |
| | 16790 | strain: DSM 16790 = |
| | | HBSQ001 |
| Hwa Pol-II-2 | Haloquadratum walsbyi DSM | Halophile, taxon: 362976, |
| | 16790 | strain: DSM 16790 = |
| | | HBSQ001 |
| Hwa PolB-1 | Haloquadratum walsbyi DSM | Halophile, taxon: 362976, |
| | 16790 | strain: DSM 16790 = |
| | | HBSQ001 |
| Hwa PolB-2 | Haloquadratum walsbyi DSM | Halophile, taxon: 362976, |
| | 16790 | strain: DSM 16790 = |
| | | HBSQ001 |
| Hwa PolB-3 | Haloquadratum walsbyi DSM | Halophile, taxon: 362976, |
| | 16790 | strain: DSM 16790 = |
| | | HBSQ001 |
| Hwa RCF | Haloquadratum walsbyi DSM | Halophile, taxon: 362976, |
| | 16790 | strain: DSM 16790 = |
| | | HBSQ001 |
| Hwa RIR1-1 | Haloquadratum walsbyi DSM | Halophile, taxon: 362976, |
| | 16790 | strain: DSM 16790 = |
| | | HBSQ001 |
| Hwa RIR1-2 | Haloquadratum walsbyi DSM | Halophile, taxon: 362976, |
| | 16790 | strain: DSM 16790 = |
| | | HBSQ001 |
| Hwa Top6B | Haloquadratum walsbyi DSM | Halophile, taxon: 362976, |
| | 16790 | strain: DSM 16790 = |
| | | HBSQ001 |
| Hwa rPol A″ | Haloquadratum walsbyi DSM | Halophile, taxon: 362976, |
| | 16790 | strain: DSM 16790 = |
| | | HBSQ001 |
| Maeo Pol-II | Methanococcus aeolicus Nankai-3 | taxon: 419665 |
| Maeo RFC | Methanococcus aeolicus Nankai-3 | taxon: 419665 |
| Maeo RNR | Methanococcus aeolicus Nankai-3 | taxon: 419665 |
| Maeo-N3 Helicase | Methanococcus aeolicus Nankai-3 | taxon: 419665 |
| Maeo-N3 RtcB | Methanococcus aeolicus Nankai-3 | taxon: 419665 |
| Maeo-N3 UDP GD | Methanococcus aeolicus Nankai-3 | taxon: 419665 |
| Mein-ME PEP | Methanocaldococcus infernus ME | thermophile, Taxon: 573063 |
| Mein-ME RFC | Methanocaldococcus infernus ME | Taxon: 573063 |
| Memar MCM2 | Methanoculleus marisnigri JR1 | taxon: 368407 |
| Memar Pol-II | Methanoculleus marisnigri JR1 | taxon: 368407 |
| Mesp-FS406 PolB-1 | Methanocaldococcus sp. FS406-22 | Taxon: 644281 |
| Mesp-FS406 PolB-2 | Methanocaldococcus sp. FS406-22 | Taxon: 644281 |
| Mesp-FS406 PolB-3 | Methanocaldococcus sp. FS406-22 | Taxon: 644281 |
| Mesp-FS406-22 LHR | Methanocaldococcus sp. FS406-22 | Taxon: 644281 |
| Mfe-AG86 Pol-1 | Methanocaldococcus fervens AG86 | Taxon: 573064 |
| Mfe-AG86 Pol-2 | Methanocaldococcus fervens AG86 | Taxon: 573064 |
| Mhu Pol-II | Methanospirillum hungateii JF-1 | taxon 323259 |
| Mja GF-6P | Methanococcus jannaschii | Thermophile, DSM 2661, |
| | (Methanocaldococcus jannaschii | taxon: 2190 |
| | DSM 2661) | |
| Mja Helicase | Methanococcus jannaschii | Thermophile, DSM 2661, |
| | (Methanocaldococcus jannaschii | taxon: 2190 |
| | DSM 2661) | |
| Mja Hyp-1 | Methanococcus jannaschii | Thermophile, DSM 2661, |
| | (Methanocaldococcus jannaschii | taxon: 2190 |
| | DSM 2661) | |
| Mja IF2 | Methanococcus jannaschii | Thermophile, DSM 2661, |
| | (Methanocaldococcus jannaschii | taxon: 2190 |
| | DSM 2661) | |
| Mja KlbA | Methanococcus jannaschii | Thermophile, DSM 2661, |
| | (Methanocaldococcus jannaschii | taxon: 2190 |
| | DSM 2661) | |
| Mja PEP | Methanococcus jannaschii | Thermophile, DSM 2661, |
| | (Methanocaldococcus jannaschii | taxon: 2190 |
| | DSM 2661) | |
| Mja Pol-1 | Methanococcus jannaschii | Thermophile, DSM 2661, |
| | (Methanocaldococcus jannaschii | taxon: 2190 |
| | DSM 2661) | |
| Mja Pol-2 | Methanococcus jannaschii | Thermophile, DSM 2661, |
| | (Methanocaldococcus jannaschii | taxon: 2190 |
| | DSM 2661) | |
| Mja RFC-1 | Methanococcus jannaschii | Thermophile, DSM 2661, |
| | (Methanocaldococcus jannaschii | taxon: 2190 |
| | DSM 2661) | |
| Mja RFC-2 | Methanococcus jannaschii | Thermophile, DSM 2661, |
| | (Methanocaldococcus jannaschii | taxon: 2190 |
| | DSM 2661) | |
| Mja RFC-3 | Methanococcus jannaschii | Thermophile, DSM 2661, |
| | (Methanocaldococcus jannaschii | taxon: 2190 |
| | DSM 2661) | |
| Mja RNR-1 | Methanococcus jannaschii | Thermophile, DSM 2661, |
| | (Methanocaldococcus jannaschii | taxon: 2190 |
| | DSM 2661) | |
| Mja RNR-2 | Methanococcus jannaschii | Thermophile, DSM 2661, |
| | (Methanocaldococcus jannaschii | taxon: 2190 |
| | DSM 2661) | |
| Mja RtcB | Methanococcus jannaschii | Thermophile, DSM 2661, |
| (Mja Hyp-2) | (Methanocaldococcus jannaschii | taxon: 2190 |
| | DSM 2661) | |
| Mja TFIIB | Methanococcus jannaschii | Thermophile, DSM 2661, |
| | (Methanocaldococcus jannaschii | taxon: 2190 |
| | DSM 2661) | |
| Mja UDP GD | Methanococcus jannaschii | Thermophile, DSM 2661, |
| | (Methanocaldococcus jannaschii | taxon: 2190 |
| | DSM 2661) | |
| Mja r-Gyr | Methanococcus jannaschii | Thermophile, DSM 2661, |
| | (Methanocaldococcus jannaschii | taxon: 2190 |
| | DSM 2661) | |
| Mja rPol A′ | Methanococcus jannaschii | Thermophile, DSM 2661, |
| | (Methanocaldococcus jannaschii | taxon: 2190 |
| | DSM 2661) | |
| Mja rPol A″ | Methanococcus jannaschii | Thermophile, DSM 2661, |
| | (Methanocaldococcus jannaschii | taxon: 2190 |
| | DSM 2661) | |
| Mka CDC48 | Methanopyrus kandleri AV19 | Thermophile, taxon: 190192 |
| Mka EF2 | Methanopyrus kandleri AV19 | Thermophile, taxon: 190192 |
| Mka RFC | Methanopyrus kandleri AV19 | Thermophile, taxon: 190192 |
| Mka RtcB | Methanopyrus kandleri AV19 | Thermophile, taxon: 190192 |
| Mka VatB | Methanopyrus kandleri AV19 | Thermophile, taxon: 190192 |
| Mth RIR1 | Methanothermobacter | Thermophile, delta H strain |
| | thermautotrophicus | |
| | (Methanobacterium | |
| | thermoautotrophicum) | |
| Mvu-M7 Helicase | Methanocaldococcus vulcanius M7 | Taxon: 579137 |
| Mvu-M7 Pol-1 | Methanocaldococcus vulcanius M7 | Taxon: 579137 |
| Mvu-M7 Pol-2 | Methanocaldococcus vulcanius M7 | Taxon: 579137 |
| Mvu-M7 Pol-3 | Methanocaldococcus vulcanius M7 | Taxon: 579137 |
| Mvu-M7 UDP GD | Methanocaldococcus vulcanius M7 | Taxon: 579137 |
| Neq Pol-c | Nanoarchaeum equitans Kin4-M | Thermophile, taxon: 228908 |
| Neq Pol-n | Nanoarchaeum equitans Kin4-M | Thermophile, taxon: 228908 |
| Nma-ATCC43099 | Natrialba magadii ATCC 43099 | Taxon: 547559 |
| MCM | | |
| Nma-ATCC43099 | Natrialba magadii ATCC 43099 | Taxon: 547559 |
| PolB-1 | | |
| Nma-ATCC43099 | Natrialba magadii ATCC 43099 | Taxon: 547559 |
| PolB-2 | | |
| Nph CDC21 | Natronomonas pharaonis DSM 2160 | taxon: 348780 |
| Nph PolB-1 | Natronomonas pharaonis DSM 2160 | taxon: 348780 |
| Nph PolB-2 | Natronomonas pharaonis DSM 2160 | taxon: 348780 |
| Nph rPol A″ | Natronomonas pharaonis DSM 2160 | taxon: 348780 |
| Pab CDC21-1 | Pyrococcus abyssi | Thermophile, strain Orsay, |
| | | taxon: 29292 |
| Pab CDC21-2 | Pyrococcus abyssi | Thermophile, strain Orsay, |
| | | taxon: 29292 |
| Pab IF2 | Pyrococcus abyssi | Thermophile, strain Orsay, |
| | | taxon: 29292 |
| Pab KlbA | Pyrococcus abyssi | Thermophile, strain Orsay, |
| | | taxon: 29292 |
| Pab Lon | Pyrococcus abyssi | Thermophile, strain Orsay, |
| | | taxon: 29292 |
| Pab Moaa | Pyrococcus abyssi | Thermophile, strain Orsay, |
| | | taxon: 29292 |
| Pab Pol-II | Pyrococcus abyssi | Thermophile, strain Orsay, |
| | | taxon: 29292 |
| Pab RFC-1 | Pyrococcus abyssi | Thermophile, strain Orsay, |
| | | taxon: 29292 |
| Pab RFC-2 | Pyrococcus abyssi | Thermophile, strain Orsay, |
| | | taxon: 29292 |
| Pab RIR1-1 | Pyrococcus abyssi | Thermophile, strain Orsay, |
| | | taxon: 29292 |
| Pab RIR1-2 | Pyrococcus abyssi | Thermophile, strain Orsay, |
| | | taxon: 29292 |
| Pab RIR1-3 | Pyrococcus abyssi | Thermophile, strain Orsay, |
| | | taxon: 29292 |
| Pab RtcB | Pyrococcus abyssi | Thermophile, strain Orsay, |
| (Pab Hyp-2) | | taxon: 29292 |
| Pab VMA | Pyrococcus abyssi | Thermophile, strain Orsay, |
| | | taxon: 29292 |
| Par RIR1 | Pyrobaculum arsenaticum DSM 13514 | taxon: 340102 |
| Pfu CDC21 | Pyrococcus furiosus | Thermophile, taxon: 186497, |
| | | DSM3638 |
| Pfu IF2 | Pyrococcus furiosus | Thermophile, taxon: 186497, |
| | | DSM3638 |
| Pfu KlbA | Pyrococcus furiosus | Thermophile, taxon: 186497, |
| | | DSM3638 |
| Pfu Lon | Pyrococcus furiosus | Thermophile, taxon: 186497, |
| | | DSM3638 |
| Pfu RFC | Pyrococcus furiosus | Thermophile, DSM3638, |
| | | taxon: 186497 |
| Pfu RIR1-1 | Pyrococcus furiosus | Thermophile, taxon: 186497, |
| | | DSM3638 |
| Pfu RIR1-2 | Pyrococcus furiosus | Thermophile, taxon: 186497, |
| | | DSM3638 |
| Pfu RtcB | Pyrococcus furiosus | Thermophile, taxon: 186497, |
| (Pfu Hyp-2) | | DSM3638 |
| Pfu TopA | Pyrococcus furiosus | Thermophile, taxon: 186497, |
| | | DSM3638 |
| Pfu VMA | Pyrococcus furiosus | Thermophile, taxon: 186497, |
| | | DSM3638 |
| Pho CDC21-1 | Pyrococcus horikoshii OT3 | Thermophile, taxon: 53953 |
| Pho CDC21-2 | Pyrococcus horikoshii OT3 | Thermophile, taxon: 53953 |
| Pho IF2 | Pyrococcus horikoshii OT3 | Thermophile, taxon: 53953 |
| Pho KlbA | Pyrococcus horikoshii OT3 | Thermophile, taxon: 53953 |
| Pho LHR | Pyrococcus horikoshii OT3 | Thermophile, taxon: 53953 |
| Pho Lon | Pyrococcus horikoshii OT3 | Thermophile, taxon: 53953 |
| Pho Pol I | Pyrococcus horikoshii OT3 | Thermophile, taxon: 53953 |
| Pho Pol-II | Pyrococcus horikoshii OT3 | Thermophile, taxon: 53953 |
| Pho RFC | Pyrococcus horikoshii OT3 | Thermophile, taxon: 53953 |
| Pho RIR1 | Pyrococcus horikoshii OT3 | Thermophile, taxon: 53953 |
| Pho RadA | Pyrococcus horikoshii OT3 | Thermophile, taxon: 53953 |
| Pho RtcB | Pyrococcus horikoshii OT3 | Thermophile, taxon: 53953 |
| (Pho Hyp-2) | | |
| Pho VMA | Pyrococcus horikoshii OT3 | Thermophile, taxon: 53953 |
| Pho r-Gyr | Pyrococcus horikoshii OT3 | Thermophile, taxon: 53953 |
| Psp-GBD Pol | Pyrococcus species GB-D | Thermophile |
| Pto VMA | Picrophilus torridus, DSM 9790 | DSM 9790, taxon: 263820, |
| | | Thermoacidophile |
| Smar 1471 | Staphylothermus marinus F1 | taxon: 399550 |
| Smar MCM2 | Staphylothermus marinus F1 | taxon: 399550 |
| Tac-ATCC25905 | Thermoplasma acidophilum, ATCC | Thermophile, taxon: 2303 |
| VMA | 25905 | |
| Tac-DSM1728 VMA | Thermoplasma acidophilum, | Thermophile, taxon: 2303 |
| | DSM1728 | |
| Tag Pol-1 | Thermococcus aggregans | Thermophile, taxon: 110163 |
| (Tsp-TY Pol-1) | | |
| Tag Pol-2 | Thermococcus aggregans | Thermophile, taxon: 110163 |
| (Tsp-TY Pol-2) | | |
| Tag Pol-3 | Thermococcus aggregans | Thermophile, taxon: 110163 |
| (Tsp-TY Pol-3) | | |
| Tba Pol-II | Thermococcus barophilus MP | taxon: 391623 |
| Tfu Pol-1 | Thermococcus fumicolans | Thermophilem, taxon: 46540 |
| Tfu Pol-2 | Thermococcus fumicolans | Thermophile, taxon: 46540 |
| Thy Pol-1 | Thermococcus hydrothermalis | Thermophile, taxon: 46539 |
| Thy Pol-2 | Thermococcus hydrothermalis | Thermophile, taxon: 46539 |
| Tko CDC21-1 | Thermococcus kodakaraensis | Thermophile, taxon: 69014 |
| | KOD1 | |
| Tko CDC21-2 | Thermococcus kodakaraensis | Thermophile, taxon: 69014 |
| | KOD1 | |
| Tko Helicase | Thermococcus kodakaraensis | Thermophile, taxon: 69014 |
| | KOD1 | |
| Tko IF2 | Thermococcus kodakaraensis | Thermophile, taxon: 69014 |
| | KOD1 | |
| Tko KlbA | Thermococcus kodakaraensis | Thermophile, taxon: 69014 |
| | KOD1 | |
| Tko LHR | Thermococcus kodakaraensis | Thermophile, taxon: 69014 |
| | KOD1 | |
| Tko Pol-1 | Pyrococcus/Thermococcus | Thermophile, taxon: 69014 |
| (Pko Pol-1) | kodakaraensis KOD1 | |
| Tko Pol-2 | Pyrococcus/Thermococcus | Thermophile, taxon: 69014 |
| (Pko Pol-2) | kodakaraensis KOD1 | |
| Tko Pol-II | Thermococcus kodakaraensis | Thermophile, taxon: 69014 |
| | KOD1 | |
| Tko RFC | Thermococcus kodakaraensis | Thermophile, taxon: 69014 |
| | KOD1 | |
| Tko RIR1-1 | Thermococcus kodakaraensis | Thermophile, taxon: 69014 |
| | KOD1 | |
| Tko RIR1-2 | Thermococcus kodakaraensis | Thermophile, taxon: 69014 |
| | KOD1 | |
| Tko RadA | Thermococcus kodakaraensis | Thermophile, taxon: 69014 |
| | KOD1 | |
| Tko TopA | Thermococcus kodakaraensis | Thermophile, taxon: 69014 |
| | KOD1 | |
| Tko r-Gyr | Thermococcus kodakaraensis | Thermophile, taxon: 69014 |
| | KOD1 | |
| Tli Pol-1 | Thermococcus litoralis | Thermophile, taxon: 2265 |
| Tli Pol-2 | Thermococcus litoralis | Thermophile, taxon: 2265 |
| Tma Pol | Thermococcus marinus | taxon: 187879 |
| Ton-NA1 LHR | Thermococcus onnurineus NA1 | Taxon: 523850 |
| Ton-NA1 Pol | Thermococcus onnurineus NA1 | taxon: 342948 |
| Tpe Pol | Thermococcus peptonophilus strain SM2 | taxon: 32644 |
| Tsi-MM739 Lon | Thermococcus sibiricus MM 739 | Thermophile, Taxon: 604354 |
| Tsi-MM739 Pol-1 | Thermococcus sibiricus MM 739 | Taxon: 604354 |
| Tsi-MM739 Pol-2 | Thermococcus sibiricus MM 739 | Taxon: 604354 |
| Tsi-MM739 RFC | Thermococcus sibiricus MM 739 | Taxon: 604354 |
| Tsp AM4 RtcB | Thermococcus sp. AM4 | Taxon: 246969 |
| Tsp-AM4 LHR | Thermococcus sp. AM4 | Taxon: 246969 |
| Tsp-AM4 Lon | Thermococcus sp. AM4 | Taxon: 246969 |
| Tsp-AM4 RIR1 | Thermococcus sp. AM4 | Taxon: 246969 |
| Tsp-GE8 Pol-1 | Thermococcus species GE8 | Thermophile, taxon: 105583 |
| Tsp-GE8 Pol-2 | Thermococcus species GE8 | Thermophile, taxon: 105583 |
| Tsp-GT Pol-1 | Thermococcus species GT | taxon: 370106 |
| Tsp-GT Pol-2 | Thermococcus species GT | taxon: 370106 |
| Tsp-OGL-20P Pol | Thermococcus sp. OGL-20P | taxon: 277988 |
| Tthi Pol | Thermococcus thioreducens | Hyperthermophile |
| Tvo VMA | Thermoplasma volcanium GSS1 | Thermophile, taxon: 50339 |
| Tzi Pol | Thermococcus zilligii | taxon: 54076 |
| Unc-ERS PFL | uncultured archaeon Gzfos13E1 | isolation_source = “Eel River |
| | | sediment”, |
| | | clone = “GZfos13E1”, |
| | | taxon: 285397 |
| Unc-ERS RIR1 | uncultured archaeon GZfos9C4 | isolation source = “Eel River |
| | | sediment”, taxon: 285366, |
| | | clone = “GZfos9C4” |
| Unc-ERS RNR | uncultured archaeon GZfos10C7 | isolation source = “Eel River |
| | | sediment”, |
| | | clone = “GZfos10C7”, |
| | | taxon: 285400 |
| Unc-MetRFS MCM2 | uncultured archaeon (Rice Cluster I) | Enriched methanogenic |
| | | consortium from rice field |
| | | soil, taxon: 198240 |

The split inteins of the disclosed compositions or that can be used in the disclosed methods can be modified, or mutated, inteins. A modified intein can comprise modifications to the N-terminal intein segment, the C-terminal intein segment, or both. The modifications can include additional amino acids at the N-terminus the C-terminus of either portion of the split intein, or can be within the either portion of the split intein. Table 2 shows a list of amino acids, their abbreviations, polarity, and charge.

TABLE 2
List of Amino Acids

| | 3-Letter | 1-Letter | | |
|---|---|---|---|---|
| Amino Acid | Code | Code | Polarity | Charge |
| Alanine | Ala | A | nonpolar | neutral |
| Arginine | Arg | R | Basic polar | positive |
| Asparagine | Asn | N | polar | neutral |
| Aspartic acid | Asp | D | acidic polar | negative |
| Cysteine | Cys | C | nonpolar | neutral |
| Glutamic acid | Glu | E | acidic polar | negative |
| Glutamine | Gln | Q | polar | neutral |
| Glycine | Gly | G | nonpolar | neutral |
| Histidine | His | H | Basic polar | Positive (10%) |
| | | | | Neutral (90%) |
| Isoleucine | Ile | I | nonpolar | neutral |
| Leucine | Leu | L | nonpolar | neutral |
| Lysine | Lys | K | Basic polar | positive |
| Methionine | Met | M | nonpolar | neutral |
| Phenylalanine | Phe | F | nonpolar | neutral |
| Proline | Pro | P | nonpolar | neutral |
| Serine | Ser | S | polar | neutral |
| Threonine | Thr | T | polar | neutral |
| Tryptophan | Trp | W | nonpolar | neutral |
| Tyrosine | Tyr | Y | polar | neutral |
| Valine | Val | V | nonpolar | neutral |

Preferably, the invention provides an N-intein protein variant of the native N-intein domain of Nostoc punctiforme (Npu) wherein the native N-intein domain has the following sequence:

(SEQ ID NO: 1)
CLSYETEILTVEYGLLPIGKIVEKRIECTVYSVDNNGNIYTQPVAQWHDR
GEQEVFEYCLEDGSLIRATKDHKFMTVDGQMLPIDEIFERELDLMRV

wherein the protein variant comprises an amino acid substitution of the asparagine (N) at position 36 of SEQ ID NO: 1 with an amino acid that increases alkaline stability of the N-intein protein variant as compared to alkaline stability of the native N-intein of SEQ ID NO:1.

Preferably, the invention provides an N-intein protein variant of SEQ ID NO: 1 wherein the protein variant comprises an amino acid substitution of the cysteine (C) at position 1 of SEQ ID NO: 1 to any other amino acid that is not cysteine in addition to an amino acid substitution of the asparagine (N) at position 36 of SEQ ID NO: 1 with an amino acid that increases alkaline stability of the N-intein protein variant as compared to alkaline stability of the native N-intein of SEQ ID NO:1.

The invention also provides an N-intein protein variant of a reference protein wherein the reference protein has at least about 50%, 60%, 75%, 80%, 85%, 90%, 95%, 96%, 97%, 98%, or 99% identity with SEQ ID NO: 1 and preferably wherein the reference protein has at least 85%, 90%, 95%, 96%, 97%, 98%, or 99% identity with SEQ ID NO: 1, and wherein the N-intein protein variant of the invention comprises an amino acid substitution of the asparagine (N) at position 36 of the reference protein with an amino acid that increases alkaline stability of the N-intein protein variant as compared to alkaline stability of the native N-intein of SEQ ID NO: 1.

In another embodiment the N-intein comprises the amino acid sequence of SEQ ID NO: 2 which is a N-intein consensus derived sequence. An N-intein variant sequences based on SEQ ID NO: 2 also comprise an amino acid at position 36 other than N that increases alkaline stability of the N-intein protein variant as compared to alkaline stability of the native N-intein of SEQ ID NO: 1. Preferably the amino acid that increases stability alkaline stability is an amino acid that are less sensitive to deamidation as compared to aparagine (N). The amino acid sequence of SEQ I D NO: 2 is as follows:

(SEQ ID NO: 2)
ALSYDTEILTVEYGFLPIGXIVEEXIEXTVYSVDXXGFVYTQPIAQWHNR
GEQEVFEYXLEDGSIIRATXDHXFMTTDGXMLPIDEIFEXGLDLXQV

wherein
X in positions 20, 35, 70, 73, and 95 are each independently selected from K, R or A;
X in position 28 is C, A or S;
X in position 36 is N, H or Q;
X in position 25 is N or R;
X is position 59 is D or C;
X in position 80 is E or Q; and
X in position 90 is Q, R or K.

Preferred embodiments of N-inteins in accordance with the invention are selected from the group of N-intein variants referred to herein as A48, B22, B72 and A41 wherein:

A48 has the sequence of of SEQ ID NO: 2 wherein:
X in positions 20, 35, 70, 73, and 95 is R;
X in position 28 is A;
X in position 36 is H;
X in position 25 is N;
X in position 59 is D;
X in position 80 is E; and
X in position 90 is Q;
   B22 has the sequence of SEQ ID NO: 2, wherein:
X in positions 20, 35, 70, 73, and 95 is A;
X in position 28 is A;
X in position 36 is H;
X in position 25 is N;
X in position 59 is D;
X in position 80 is E; and
X in position 90 is Q;
   B72 has the sequence of SEQ ID NO: 2, wherein:
X in positions 20, 35, 70, 73, and 95 is K;
X in position 28 is C;
X in position 36 is H;
X in position 25 is N;
X in position 59 is D;
X in position 80 is E; and
X in position 90 is Q
   A40 has the sequence of SEQ ID NO: 2, wherein:
X in position 20, 35, 70, 73, and 95 is R;
X in position 28 is A;
X in position 36 is N;
X in position 25 is N;
X in position 59 is D;
X in position 80 is E; and
X in position 90 is Q.
   A41 has the sequence of SEQ ID NO: 2, wherein:
X in positions 20, 35, 70, 73, and 95 is K;
X in position 28 is A;
X in position 36 is N;
X in position 25 is N;
X in position 59 is D;
X in position 80 is E; and
X in position 90 is Q;
   Comparative ligand A53, has the sequence of SEQ ID NO: 2 wherein:
X in positions 20, 35, 70, 73, and 95 is K;
X in position 28 is C;
X in position 36 is N;
X in position 25 is N;
X in position 59 is D;
X in position 80 is E; and
X in position 90 is Q.

The N-intein of the invention may be coupled to solid phase, such as a membrane, fiber, particle, bead or chip. The solid phase may be a chromatography resin of natural or synthetic origin, such as a natural or synthetic resin, preferably a polysaccharide such as agarose. The solid phase, such as a chromatography resin, may be provided with embedded magnetic particles. In another embodiment the solid phase is a non-diffusion limited resin/fibrous material.

In this case the solid phase may be formed from one or more polymeric nanofibre substrates, such as electrospun polymer nanofibres. Polymer nanofibres for use in the present invention typically have mean diameters from 10 nm to 1000 nm. The length of polymer nanofibres is not particularly limited. The polymer nanofibres can suitably be monofilament nanofibres and may e.g. have a circular, ellipsoidal or essentially circular/ellipsoidal cross section. Typically, the one or more polymer nanofibres are provided in the form of one or more non-woven sheets, each comprising one or more polymer nanofibers. A non-woven sheet comprising one or more polymer nanofibres is a mat of said one or more polymer nanofibres with each nanofibre oriented essentially randomly, i.e. it has not been fabricated so that the nanofibre or nanofibres adopts a particular pattern. Non-woven sheets typically have area densities from 1 to 40 g/m2. Non-woven sheets typically have a thickness from 5 to 120 μm. The polymer should be a polymer suitable for use as a chromatography medium, i.e. an adsorbent, in a chromatography method. Suitable polymers include polyamides such as nylon, polyacrylic acid, polymethacrylic acid, polyacrylonitrile, polystyrene, polysulfones e.g. polyethersulfone (PES), polycaprolactone, collagen, chitosan, polyethylene oxide, agarose, agarose acetate, cellulose, cellulose acetate, and combinations thereof.

The N-intein according to the invention may be immobilized on a solid support in a very high degree, 0.2-2 μmole/ml N-intein is coupled per ml resin (swollen gel).

The N-intein according to the invention may be coupled to the solid phase via a Lys-tail, comprising one or more Lys, such as at least two, on the C-terminal. Alternatively, the N-intein is coupled to the solid phase via a Cys-tail on the C-terminal.

### C-intein Protein Variants

Preferably the invention also provides a C-intein comprising the following sequence SEQ ID NO 3 as follows:

(SEQ ID NO: 3)
VKIVSRKSLGVQNVYDIGVEKDHNFLLANGLIASN

or sequences having at least 50%, 60%, 75%, 80%, 85%, 90%, 95%, 96%, 97%, 98%, or 99% identity therewith and preferably sequences having at least 85%, 90%, 95%, 96%, 97%, 98%, or 99% identity therewith.

It will be appreciated that selection of the N-intein and C-intein can be from the same wild type split intein (e.g., both from Npu, or a variant of either the N- or C-intein, or alternatively can be selected from different wild type split inteins or the consensus split intein sequences, as it has been discovered that the affinity of a N-fragment for a different C-fragment (e.g., Npu N-fragment or variant thereof with Ssp C-fragment or variant thereof) still maintains sufficient binding affinity for use in the disclosed methods.

### Vectors Comprising Intein Variants of the Invention

In a third aspect, the invention relates to a vector comprising the above C-intein of SEQ ID NO: 3 and a gene encoding a protein of interest (POI). Also disclosed herein are vectors comprising nucleic acids encoding the C-terminal intein segment, as well as cell lines comprising said vectors. As used herein, plasmid or viral vectors are agents that transport the disclosed nucleic acids, such as those encoding a C-terminal intein segment and a peptide of interest, into a cell without degradation and include a promoter yielding expression of the gene in the cells into which they can be delivered. In one example, a C-terminal intein segment and peptide of interest are derived from either a virus or a retrovirus. Retroviral vectors are able to carry a larger genetic payload, i.e., a transgene or marker gene, than other viral vectors and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes; they are thermostable and can be stored at room temperature.

### Split Intein Systems

Preferably, the invention provides a split intein system for affinity purification of a protein of interest (POI), comprising a N-intein and C-intein as described above.

Preferably the N-intein comprises a N36H mutation for increased alkaline stability.

Preferably the N-intein is attached to a solid phase and the C-intein is co-expressed with the POI and used as a tag for affinity purification of the POI. Vice versa is also possible, ie attaching the C-intein to a solid phase and using the N-intein as a tag, but the former is preferred.

The alkaline stability of the N-intein ligand in the split intein system according to the invention enables be re-generation after cleavage of the POI from the solid phase, under alkaline conditions, such as 0.05-0.5 M NaOH. The solid phase may be regenerated up to 100 times.

In one embodiment the C-intein and an additional tag is co-expressed with the POI. The additional tag may be any conventional chromatography tag, such as an IEX tag or an affinity tag.

### Methods of Purifying a Protein of Interest (POI)

In a fifth aspect the invention relates to a method for purification of a protein of interest (POI), using the split intein system according to the invention, comprising association of the C-intein and N-intein at neutral pH, such as 6-8, and in the presence of divalent cations (which impairs spontaneous cleavage); washing said solid phase in the presence of divalent cations; addition of a chelator to allow spontaneous cleavage between C-intein and POI; collection of tagless POI; and re-generating said solid phase under alkaline conditions, such as 0.5M NaOH.

This protocol is suitable for protein non-sensitive for Zn. The advantages are long contact times are allowed with the resin and addition of large sample volume. Sample loading could be made for long times, such as up to 1.5 hours.

According to the invention more than 30% yield, preferably 50%, most preferably more than 80% of POI is achieved in less than 4 hours cleavage.

The invention enables a high ligand density when the N-intein is immobilized to a solid phase. Preferably the N-intein is attached to a chromatography resin, such as agarose or any other suitable resin for protein purification. According to the invention it is possible to achieve a static binding capacity of 0.2-2 μmole/ml C-intein bound POI per settled ml resin.

### Affinity Tags

The invention also relates to a method for purification of a protein of interest (POI), comprising the following steps: co-expressing a POI with a C-intein according to the invention and an additional tag; binding said additional tag to its binding partner on a solid phase; cleaving off the POI and the C-intein; binding said C-intein to an N-intein attached to a solid phase at neutral pH and cleaving off said bound C-intein and N-intein from said POI; and re-generating said solid phase under alkaline conditions, such as 0.5M NaOH. The purpose of this twin tag: increased purity (enables dual affinity purification), solubility, detectability.

Affinity tags can be peptide or protein sequences cloned in frame with protein coding sequences that change the protein's behavior. Affinity tags can be appended to the N- or C-terminus of proteins which can be used in methods of purifying a protein from cells. Cells expressing a peptide comprising an affinity tag can be expressed with a signal sequence in the supernatant/cell culture medium. Cells expressing a peptide comprising an affinity tag can also be pelleted, lysed, and the cell lysate applied to a column, resin or other solid support that displays a ligand to the affinity tags. The affinity tag and any fused peptides are bound to the solid support, which can also be washed several times with buffer to eliminate unbound (contaminant) proteins. A protein of interest, if attached to an affinity tag, can be eluted from the solid support via a buffer that causes the affinity tag to dissociate from the ligand resulting in a purified protein, or can be cleaved from the bound affinity tag using a soluble protease. As disclosed herein, the affinity tag is cleaved through the self-cleaving mechanism of the C-intein segment in the active intein complex.

Examples of affinity include, but are not limited to, maltose binding protein, which can bind to immobilized maltose to facilitate purification of the fused target protein; Chitin binding protein, which can bind to immobilized chitin; Glutathione S transferase, which can bind to immobilized glutathione; poly-histidine, which can bind to immobilized chelated metals; FLAG octapeptide, which can bind to immobilized anti-FLAG antibodies.

Affinity tags can also be used to facilitate the purification of a protein of interest using the disclosed modified peptides through a variety of methods, including, but not limited to, selective precipitation, ion exchange chromatography, binding to precipitation-capable ligands, dialysis (by changing the size and/or charge of the target protein) and other highly selective separation methods.

In some aspects, affinity tags can be used that do not actually bind to a ligand, but instead either selectively precipitate or act as ligands for immobilized corresponding binding domains. In these instances, the tags are more generally referred to as purification tags. For example, the ELP tag selectively precipitates under specific salt and temperature conditions, allowing fused peptides to be purified by centrifugation. Another example is the antibody Fc domain, which serves as a ligand for immobilized protein A or Protein G-binding domains.

### Proteins of Interest

Target proteins for all protocols are: any recombinant proteins, especially proteins requiring native or near native N-terminal sequences, for example therapeutic protein candidates, biologics, antibody fragments, antibody mimetics, protein scaffolds, enzymes, recombinant proteins or peptides, such as growth factors, cytokines, chemokines, hormones, antigen (viral, bacterial, yeast, mammalian) production, vaccine production, cell surface receptors, fusion proteins.

The invention will now be described more closely in association with some non-limiting examples and the accompanying drawings.

### EXAMPLES

### Experiment 1: Alkali Stability of N-intein Ligands of the Invention

The N-intein ligands A40, A41 and A48 according to the invention were immobilized on Biacore™ CM5 sensor chips (Cytiva, Sweden) in an amount sufficient to give an immobilized level of about 450 Response Units (RU) or higher. To follow the relative binding capacity of a C-intein tagged POI to the immobilized surface, 20 μg/ml C-intein (SEQ ID NO: 3) tagged Green Fluorescent Protein (GFP) was flowed over the chip for 1 minute and the signal strength was noted. The surface was then cleaned-in-place (CIP), i.e. flushed with 100 mM NaOH, 4 M Guanidine-HCl for 10 minutes at room temperature 22±3° C. This was repeated for 50 cycles and the immobilized ligand alkaline stability was followed as the relative loss of relative C-intein tagged GFP binding capacity (signal strength) after each cycle.

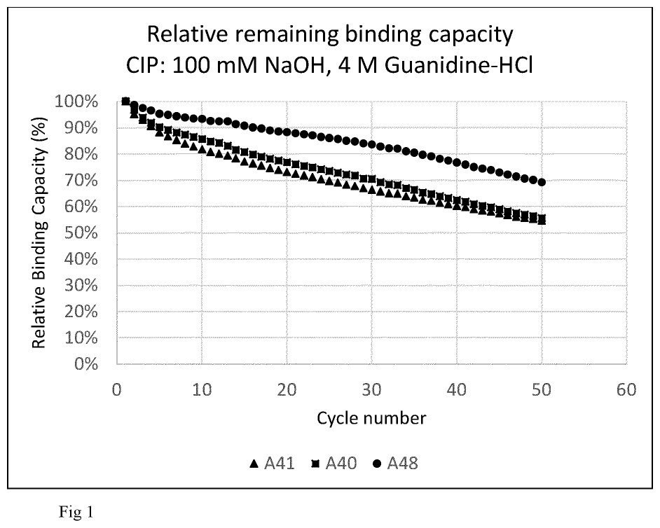

The results are shown in FIG. 1 and indicate that the ligand A48 (with the N36H mutation) has an improved alkaline stability compared to the ligands A41 and A40. The alkaline stability was further improved compared to native sequences. In addition, a N36H mutation significantly improved alkali stability as compared to wild type Npu N-intein sequence (A52 with a CIA mutation as compared to SEQ ID NO: 1).

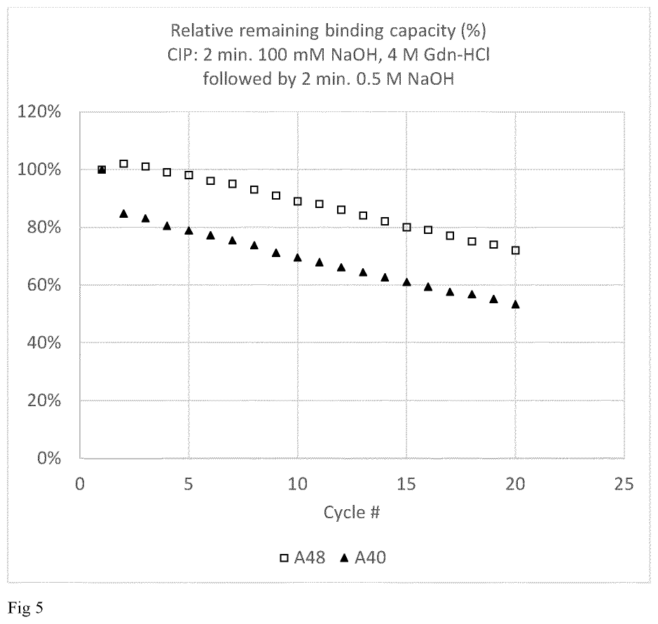

The relative remaining binding capacity after 50 CIP cycles (%) was 55% for A40 and A41 while it was 69% for A48. Alkali stability using 0.5M NaOH is shown in FIG. 5.
FIG. 5 shows the results for A40 and A48 during 20 cycles. Relative remaining binding capacity (%)
CIP: 2 min. 100 mM NaOH, 4 M Gdn-HCl, followed by 2 min. 0.5 M NaOH.

### Experiment 2: Alkali Stability of N-intein Ligands of the Invention

The purified N-intein ligands A53, B72, B22 and A48 were immobilized on Biacore™ CM5 sensor chips (Cytiva, Sweden) in an amount sufficient to give an immobilized level of about 450 Response Units (RU) or higher. To follow the relative binding capacity of an uncleavable C-intein tagged POI to the immobilized surface, 20 μg/ml uncleavable C-intein (SEQ ID NO 3) tagged IL-1b was flowed over the chip for 1 minute and the signal strength was noted. The surface was then cleaned-in-place (CIP), i.e. flushed with 100 mM NaOH, 4 M Guanidine-HCl for 10 minutes at room temperature 22±3° C. This was repeated for 50 cycles and the immobilized ligand alkaline stability was followed as the relative loss of uncleavable C-intein tagged IL-1b binding capacity (signal strength) after each cycle.

The results are shown in FIG. 2 and indicate that all three ligands with N36H mutations, (A48, B22 and B72) have improved alkaline stability compared to the ligand A53. The relative remaining binding capacity after 50 CIP cycles (%) for A53 was only 20% while it was 28% for B72, 30% for B22 and 35% for A48.

### Experiment 3: Immobilization of N-intein Ligand A48 to Agarose Gel Resin

5 millilitres epoxy activated cross-linked activated gel resin was added into a polyproylene test-tube. 2.7 millilitres, corresponding to 135 milligram N-intein ligand A48 having a C-terminal Lys-tail in phosphate buffer was added into the tube followed by addition of 1.3 millilitres of phosphate buffer (pH 12.1) to adjust the agarose resin slurry to be about 50% and then 2 gram sodium sulfate was added. The pH of the resulting reaction mixture was adjusted to 11.5. And the reaction mixture was heated up to 33° C. in a shaking table and kept shaking at 33° C. for 4 hours. Then the slurry was transferred to glass filter and washed with 10 millilitres of distilled water 3 times. After washing, the gel was transferred into the three-neck round bottom flask (RBF) and 5 millilitres of Tris buffer (pH 8.6) with 375 microlitres thioglycerol was added. The reaction mixture was at the shaking table at 45° C. for 2 hours. After the reaction, the slurry was transferred to glass filter. The gel was washed with 5 millilitres of basic wash buffer 3 times and then 5 millilitres of acidic wash buffer 3 times. Repeated this base/acid wash another 2 times, in total 18 washes in this step. Then the gel resin was washed with 5 millilitres of distilled water 10 times. The washed and drained gel was kept in 20% ethanol in fridge before analysis.

The dry weight of gel resin was determined by measuring the weight of 1 millilitre of gel. In the sample preparation, 2 gram of drained gel resin mixed well with 2 gram of water to give about 50% resin slurry and then the slurry was added into the 1 mL Teflon cube. Then vacuum was applied to drain the gel in the cube and thus 1 mL of gel was obtained. Transfer the gel onto the dry weight balance. The weight was determined after 35 minutes with drying temperature set at 105° C.

Amino acid analysis was measured after the dry weight determination. With the corresponding dry weights and information of the size and primary amino sequence of the protein the ligand density could be derived in mg/mL gel resin.

Results for the coupled agarose resin was a dry-weight of 90.6 mg/ml and with a ligand content of 18.4 mg/ml which corresponds to 1.38 umole/ml.

### Experiment 4: Static Binding Capacity in Relation to Ligand Density

The proposed capacity method presented herein can measure binding capacity of the resin in test tubes.

### Reaction Setup

Briefly, prototype resin with immobilized A48 ligand with various ligand densities and dual tagged test-protein A43 (SEQ ID NO: 5) were separately diluted in assay buffer (2× PBS) to 2.5% resin slurry and 0.4 mg/mL, respectively. 50 μL of the 2.5% resin slurry was added to an ILLUSTRA™ microspin column followed by addition of 150 μL diluted A43 (SEQ ID NO: 5). The reactions were allowed to incubate with 1450 rpm shaking at 22° C. for a 2 hour fixed timepoint before centrifuged at 3000 rcf for 1 min.

### SDS-PAGE

Centrifuged samples (containing cleaved protein and unbound non-cleaved protein) were mixed 1:1 with 2× SDS-PAGE reducing sample buffer, boiled for 5 minutes at 95° C. and subjected to SDS-PAGE (18 μL loaded). A C-intein tagged test-protein, A43 (SEQ ID NO: 5) standard was added (usually a five-point standard between 18.75-300 μg/mL) in order to be able to calculate concentrations from the densitometric volumes. Gels were coomassie stained for 60 min (˜100 mL/gel) followed by destaining for 120-180 min at room temperature with gentle agitation (until background is completely clear). Densitometric quantification of the uncleaved/unbound and cleaved test-protein was performed with the IQ TL software. The densitometric raw data was then exported to Microsoft Excel.

### SBC Calculations

Since the test-protein input in the reactions are known we can indirectly calculate the static binding capacity (SBC) by the following equation:

$$SBC \frac{mg}{mL} = \frac{( input amount in µg - unbound amount in µg )}{resin volume ( µL )}$$

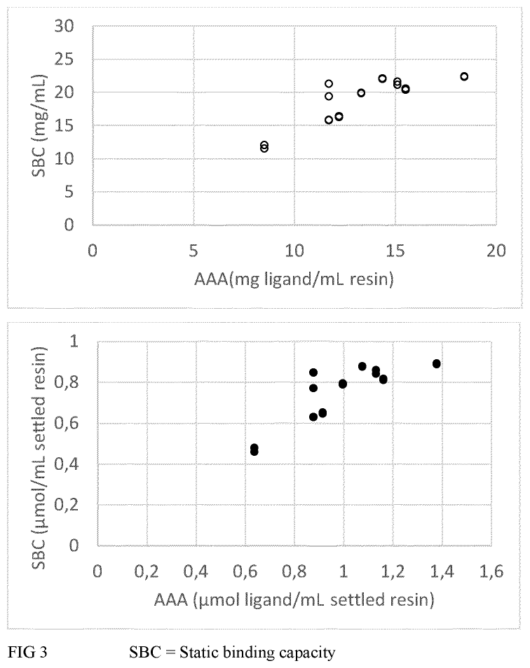

FIG. 3 shows static binding capacity of the N-intein ligands of the invention. Amino acid analysis (AAA) done by conventional method. The A48 prototypes were coupled by epoxy chemistry to porous agarose particles.

### Experiment 5: Purification of Elongation Factor G Without and With Zn Protocol

Elongation factor G, (Ef-G) from Thermoanaerobacter tengcongensis was purified in this example using a resin prototype with immobilized ligand A48. C-intein (SEQ ID NO 3) tagged EfG was expressed intracellularly in E. coli strain BL21 (DE3).

Frozen cell-pellet after fermentation harvest was thawed and resuspended with extraction buffer, (20 mM Tris-HCl, pH 8.0) by magnetic stirring. DNAse I (bovine pancreas) and 1 mM MgSO₄ was added followed by addition of lysozyme (hen egg). After stirring for 30 minutes at room temperature the resuspended and lysozyme treated cell suspension was heated in a water-bath to 70-75° C. and kept at this temperature for 5 minutes. After cooling the extract briefly on ice, the extract was clarified by centrifugation.

Purification using a Zn-free protocol was done on an ÄKTA™ Avant system at 2 ml/min during sample loading and washing and then at 1 ml/min. A 1 ml HiTrap™ column containing immobilized A48 ligand was used. Equilibration and binding of the C-intein tagged target protein was done in a 20 mM MES buffer supplemented with 100 mM NaCl at pH 6.3 and the sample was adjusted to pH 6.3 using 2M Acetic acid. Column wash after sample application and subsequent elutions were done with a 20 mM Tris-HCl buffer supplemented with 400 mM NaCl at pH 8.0. After column washing the flow was stopped for 4 hours of incubation at room temperature and then cleaved EfG was eluted. A second stop in flow was added to allow a second elution, which was done after additional 16 hours of incubation.

17.8 mg pure, tag-free EfG was eluted after 4 hours incubation on the HiTrap™ column. The mass difference between eluted protein and CIPed protein was equal to the mass of the C-intein tag according to mass spectrometry analysis. The purity according to SDS-PAGE was high as well as in SEC-analysis on Superdex™ 200 Increase. The total protein amount was calculated from the theoretical UV absorption coefficent at 280 nm and the UV-signal on diluted elution and CIP fractions.

The purification was repeated using a protocol including Zn-ions to the equilibration buffer and the clarified sample. The final Zn-concentration was 1.6 mM. The flowrate was reduced to 0.5 ml/min during sample application and then increased to 1 ml/imn during wash and elution. Wash and elution was done with a 50 mM Tris-HCl, 20 mM imidazole buffer pH 7.5. Only one elution peak was collected in this purification and that was after 4 hours of incubation after column washing.

16.6 mg pure, tag-free EfG was eluted after 4 hours incubation on the HiTrap™ column. The purity according to a SEC-analysis on Superdex™ 200 Increase was 92%. The total protein amount was calculated from the theoretical UV absorption coefficent at 280 nm and the UV-signal on diluted elution fractions.

### Experiment 6: Purification of IL-1β

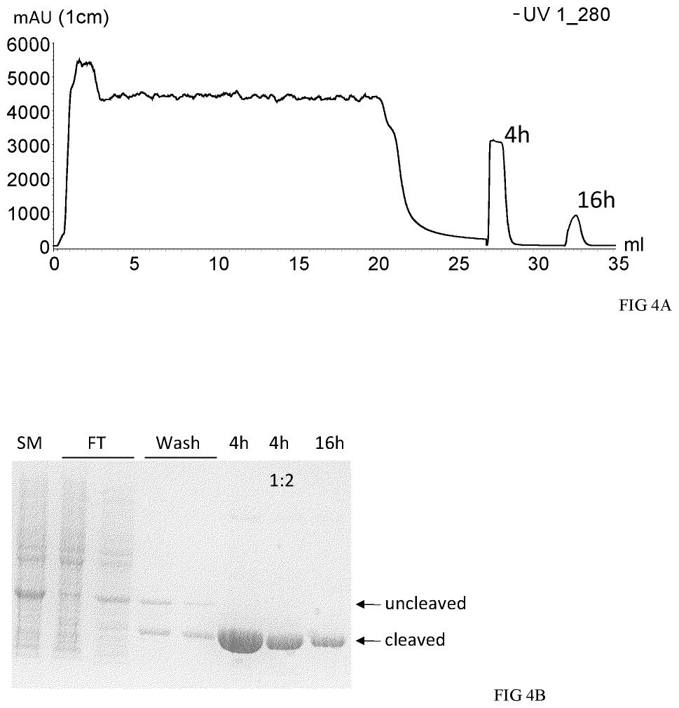

A 1 ml HiTrap™ column containing immobilized A48 ligand was used for purification of the C-intein tagged target protein IL-1β (SEQ ID NO: 5) expressed intracellularly in E. coli BL21 (DE3) and lysed by sonication. Soluble protein were harvested by centrifugation and loaded onto a 1 mL HiTrap™ column immobilized with the A48 ligand. The Zn-free protocol (as in Experiment 4) was used on an ÄKTA™ Avant system at 4 ml/min (600 cm/h linear flow rate) during sample loading and washing. The run was then paused for 4 h before initiating flow again at 1 mL/min to elute the cleaved protein (4 h cleavage fraction). The run was then paused again for an additional 12 h before starting the flow at 1 mL/min to elute the protein that had not been cleaved after 4 h. Equilibration and binding of the wash and elution was performed with one single buffer. A chromatogram from the purification is shown in FIG. 4A. The start material, flow through, wash fractions, 4 h and 16 h elution fractions were subjected to SDS-PAGE and Coomassie staining and subsequent analysis using IQTL software (FIG. 4B).

9.4 mg cleaved IL-1β was eluted after 4 hours incubation on the HiTrap™ column followed by an additional 1.1 mg after 16 h. The purity was 99.5 (4 hours) and 99.8% (16 hours) according to SDS-PAGE analysis. The total protein amount was calculated from the theoretical UV absorption coefficient of the cleaved protein at 280 nm.

### Experiment 7: Purification of Receptor Binding Domain of SARS-COV-2

The receptor binding domain (RBD) of SARS-COV-2 NCBI tagged with C-intein was expressed in ExpiHEK cells and secreted into the cell culture medium. Approximately 210 mL supernatant was loaded onto a 1 mL HiTrap column with immobilized A48 ligand and without any addition of salts or other additives to the cell culture supernatant using an ÄKTA™ Avant FPLC system. Sample application and wash was performed at 4 mL/min (load time ˜52.5 min (600 cm/h linear flow rate)) followed by 6 column volumes of wash followed by a pause/hold step for 4 h. The elution phase was performed at 1 mL/min. The column was left for additional 68 h followed by a second elution. A single 40 mM phosphate buffer pH 7.4 buffer supplemented with 300 mM NaCl was used for all chromatography steps.

The theoretical absorbance 0.1% coefficient was used to determine protein concentration and yield within the Unicorn™ software (Cytiva Sweden AB). Purity was determined by densitometric SDS-PAGE analysis. For this experiment a total of 14.1 mg cleaved protein was obtained with a purity above 96%. Theoretical molecular weight was ˜25 kDa while experimental SDS-PAGE analysis indicates a molecular weight of 33 kDa which is explained by two glycosylations and was also determined by mass spectrometry analysis.

The CCT-RBD protein has the following sequence:

(SEQ ID NO: 4)

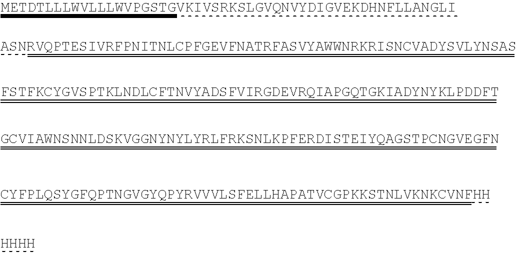

Signal sequence- bold underline.
CCT-tag- dotted underline.
RBD domain is double underlined.
His Tag- dashed underline

The purity results from the cleaved protein are found in Table 3.

TABLE 3

| Elution | cleavage time | Purity | Yield target protein |
|---|---|---|---|
| 4 h | 4 hours | 96.5% | 4.9 milligram |
| 72 h | 72 hours | 99.4% | 9.2 milligram |

### Experiment 8: Tandem Tagging and Affinity Purification on Two Columns

E. coli BL21(DE3) was transformed with the A43 expression plasmid TwinStrep™ and C-intein (SEQ ID NO 3) tagged IL-1b and plated on an agar plate containing 50 μg/ml Kanamycin. The next day, a single colony was picked and grown in 5 ml of Luria-Bertani (LB) broth to OD600 0.6. The culture was transferred to 200 ml LB broth containing the same antibiotics and grown at 37° C. until OD600 was 0.6. Protein expression was induced at 22° C. for 16 hours by the addition of Isopropyl b-D-1-thiogalactopyranoside (IPTG, 0.5 mM). After expression, the cells were harvested by centrifugation at 4,000×g for 15 minutes and stored at −80° C. until use.

For purification, the cell pellets were resuspended in Buffer A1 (100 mM Tris-HCl, 150 mM NaCl, 1 mM EDTA, pH 8.0) at 10 ml per gram wet-weight and disrupted by ultra-sonication (Sonics Vibracell, microtip, 30% amplitude, 2 sec on, 4 sec off, 3 min in total).

The supernatant containing the soluble fraction was collected after centrifugation at 40,000×g for 20 minutes at 4° C. and passed through a 5 ml HiTrap™ column, Streptactin™ XT (GE Healthcare, Sweden). The column was washed with the same Buffer A1 until the UV-absorbance at 280 nm was below 20 mAU. Bound C-intein tagged IL-1b was eluted in Buffer B1 (100 mM Tris-HCl, 150 mM NaCl, 1 mM EDTA, 50 mM Biotin, pH 8.0) and collected.

Purified protein was immediately applied to a 1 ml HiTrap™ column packed with a resin containing immobilized N-intein ligand A48 without adding the inhibitor ZnCl₂. The cleaved, tag-free IL-1b was collected in the flow-through.

(SEQ ID NO: 5)

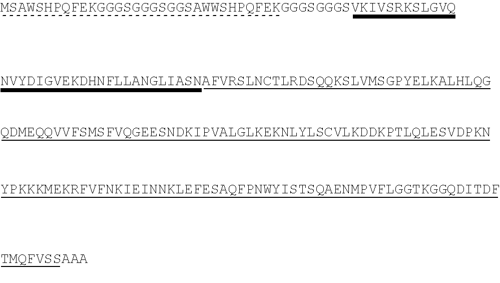

TwinStrep- dotted underlining
CCT- bold underlining
IL1b (test-protein)- underlined

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention

1. An N-intein variant comprising at least one amino acid substitution of a native split intein wherein the N-intein protein variant sequence does not include an asparagine (N) in at least position 36 as measured from the initial catalytic cysteine and wherein the substituted amino acid provides increased alkaline stability as compared to the native N-intein protein sequence or a consensus N-intein sequence.

2. The N-intein variant of claim 1, wherein the substituted amino acid that provides increased alkaline stability is histidine (H) or glutamine (Q).

3. An N-intein protein variant of the wildtype N-intein domain of Nostoc punctiforme (Npu) wherein the wildtype Npu N-intein domain comprises the following sequence:
CLSYETEILTVEYGLLPIGKIVEKRIECTVYSVDNNGNIYTQPVAQWHDRGEQEVFE YCLEDGSLIRATKDHKFMTVDGQMLPIDEIFERELDLMRV (SEQ ID NO: 1), wherein the protein variant comprises an amino acid substitution of the asparagine (N) in at least position 36 of SEQ ID NO: 1 with an amino acid that increases alkaline stability of the N-intein protein variant as compared to alkaline stability of the wildtype N-intein domain and variants or the wildtype N-intein domain.

4. The N-intein protein variant of claim 3, wherein the amino acid substitution that increases alkaline stability is histidine (H) or glutamine (Q).

5. The N-intein protein variant according to claim 4, wherein the amino acid substitution that increases alkaline stability is histidine (H).

6. An N-intein variant sequence comprising:
(SEQ ID NO: 2)
ALSYDTEILTVEYGFLPIGXIVEEXIEXTVYSVDXXGFVYTQPIAQWHNR
GEQEVFEYXLEDGSIIRATXDHXFMTTDGXMLPIDEIFEXGLDLXQV
wherein,
X in positions 20, 35, 70, 73, and 95 are each independently selected from K, R or A;
X in position 28 is C, A or S;
X in position 36 is N, H or Q;
X in position 25 is N or R;
X is position 59 is D or C;
X in position 80 is E or Q; and
X in position 90 is Q, R or K;
and wherein the alkaline stability is increased as compared to SEQ ID NO: 1.

7. The N-intein variant sequence according to claim 6, wherein
X in positions 20, 35, 70, 73, and 95 is R;
X in position 28 is A;
X in position 36 is H;
X in position 25 is N;
X in position 59 is D;
X in position 80 is E; and
X in position 90 is Q;

8. The N-intein variant sequence according to claim 6, wherein
X in positions 20, 35, 70, 73, and 95 is A;
X in position 28 is A;
X in position 36 is H;
X in position 25 is N;
X in position 59 is D;
X in position 80 is E; and
X in position 90 is Q;

9. The N-intein variant sequence according to claim 6, wherein
X in positions 20, 35, 70, 73, and 95 is K;
X in position 28 is C;
X in position 36 is H;
X in position 25 is N;
X in position 59 is D;
X in position 80 is E; and
X in position 90 is Q

10. The N-intein variant sequence according to claim 6, wherein
X in position 20, 35, 70, 73, and 95 is R;
X in position 28 is A;
X in position 36 is N;
X in position 25 is N;
X in position 59 is D;
X in position 80 is E; and
X in position 90 is Q.

11. The N-intein variant sequence according to claim 6, wherein
X in positions 20, 35, 70, 73, and 95 is K;
X in position 28 is A;
X in position 36 is N;
X in position 25 is N;
X in position 59 is D;
X in position 80 is E; and
X in position 90 is Q;

12. The N-intein variant sequence according to claim 1, which is coupled to solid phase, such as a membrane, fiber, particle, bead or chip.

13. The N-intein variant sequence according to claim 12, wherein the solid phased is a chromatography resin of natural or synthetic origin.

14. The N-intein variant sequence according to claim 12, wherein the solid phase is a chromatography resin, such as a natural or synthetic resin, preferably a polysaccharide such as agarose.

15. The N-intein variant sequence according to claim 13, wherein the solid phase is provided with embedded magnetic particles.

16. The N-intein variant sequence according to claim 12, wherein the solid phase is a non-diffusion limited resin/fibrous material.

17. The N-intein variant sequence according to claim 12, wherein the N-intein is coupled to the solid phase via a Lys-tail, comprising one or more Lys, on the C-terminal.

18. The N-intein variant sequence according to claim 12, wherein the N-intein is coupled to the solid phase via a Cys-tail on the C-terminal.

19. The N-intein variant sequence according to claim 12, wherein 0.2-2 μmole/ml N-intein is coupled per ml solid phase, preferably chromatography resin (ml swollen gel).

20. The N-intein sequence according to claim 1, wherein the N-intein is stabile under alkaline conditions corresponding to 0.05M-0.5M, preferably 0.1-0.5M NaOH.

21. A C-intein variant sequence comprising the amino acid sequence:
(SEQ ID NO: 3)
VKIVSRKSLGVQNVYDIGVEKDHNFLLANGLIASN
or sequences having at least 85% identity therewith.

22. A vector comprising the C-intein according to claim 21, and a gene encoding a protein of interest (POI).

23. A split intein system for affinity purification of a protein of interest (POI), comprising a N-intein variant sequence of a native N-intein and a C-intein, wherein the N-intein variant sequence has a N36H or N36Q mutation as compared to native N-intein.

24. A Split intein system according to claim 23, comprising a N-intein sequence variant of claim 1 and a C intein variant sequence of SEQ ID NO: 3.

25. A split intein system according to claim 23, wherein the C-intein and an additional tag is co-expressed with the POI.

26. A split intein system according to claim 23, wherein the N-intein is immobilized to a solid phase and the solid phase is re-generated after cleavage of the POI from the solid phase.

27. A split intein system according to claim 26, wherein the solid phase is re-generated under alkaline conditions, such as 0.05-0.5 M NaOH.

28. A split intein system according to claim 26, wherein the solid phase is regenerated up to 100 cycles, such as up to 50 cycles.

29. A chromatography column comprising a chromatography resin which comprises one or more N-intein variant sequence ligands, wherein the N-intein variant sequence is as defined in claim 1.

30. A method for purification of a C-intein tagged protein of interest (POI), using the split intein system according to claim 23, wherein the N-intein is immobilized to a solid phase; comprising contacting the C-intein and N-intein at neutral pH, such as 6-8, and in the presence of divalent cations; washing said solid phase in the presence of divalent cations; addition of a chelator to allow spontaneous cleavage between C-intein and POI; collection of tagless POI; and re-generating said solid phase under alkaline conditions, such as 0.05-0.5M NaOH.

31. The method for purification of a C-intein tagged protein of interest (POI), using the split intein system according to claim 23, wherein the N-intein is immobilized to a solid phase; comprising contacting the C-intein and N-intein at neutral pH, such as 6-8, preferably under high flow rate; washing said solid phase; collection of tagless POI after cleavage between C-intein and POI; and re-generating said solid phase under alkaline conditions, such as 0.05-0.5M NaOH.

32. A method for purification of a protein of interest (POI), comprising the following steps: co-expressing a POI with a C-intein according SEQ ID NO 3 and an additional tag; binding said additional tag to its binding partner on a first solid phase; cleaving off the POI and the C-intein; binding said C-intein to an N-intein attached to a second solid phase at neutral pH and cleaving off said bound C-intein and N-intein from said POI; and re-generating said second solid phase under alkaline conditions, such as 0.05-0.5M NaOH.

33. The method according to claim 32, wherein the additional tag is an affinity tag, ion exchange, hydrophobic interaction, solubility, multimodal.

34. The method according to claim 30, the alkaline conditions are combined with chaotrope agents, such as guanidine or urea, and the solid phase may be regenerated up to 100 times.

35. The method according to claim 30, wherein the POI's are: proteins requiring native or near native N-terminal sequences, for example therapeutic protein candidates, biologics, antibody fragments, antibody mimetics, enzymes, recombinant proteins or peptides, such as growth factors, cytokines, chemokines, hormones, antigen (viral, bacterial, yeast, mammalian) production, vaccine production, cell surface receptors, fusion proteins.

36. The method according to claim 30, wherein more than 30%, preferably more than 50%, most preferably more than 80% yield of POI is achieved in less than 4 hours cleavage.

37. The method according to claim 30, wherein the N-intein is immobilized on a chromatography resin, and wherein the static binding capacity is 0.2-2 μmole/ml C-intein bound POI per settled ml resin.

38. An N-intein variant according to claim 1, wherein all asparagine (N) amino acid residues are substituted with amino acid residue that provides increased alkaline stability as compared to the native N-intein protein sequence.

39. An N-intein variant according to claim 1, wherein all asparagine (N) amino acid residues are substituted with amino acid residue that provides increased alkaline stability and wherein the cysteine at the first residue is substituted with any other amino acid.


SEQUENCE LISTING (5 sequences)

SEQ ID NO: 1 - protein, 97 aa, Nostoc punctiforme
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRV

SEQ ID NO: 2 - protein, 97 aa, Artificial Sequence
  source: /note=“Description of Artificial Sequence Synthetic polypeptide”
  VARIANT (20)..(20): /replace=“R” or “A”
  VARIANT (25)..(25): /replace=“R”
  VARIANT (28)..(28): /replace=“A” or “S”
  VARIANT (35)..(35): /replace=“R” or “A”
  VARIANT (36)..(36): /replace=“H” or “Q”
  VARIANT (59)..(59): /replace=“C”
  VARIANT (70)..(70): /replace=“R” or “A”
  VARIANT (73)..(73): /replace=“R” or “A”
  VARIANT (80)..(80): /replace=“Q”
  VARIANT (90)..(90): /replace=“R” or “K”
  VARIANT (95)..(95): /replace=“R” or “A”
  SITE (1)..(97): /note=“Variant residues given in the sequence have no preference with respect to those in the annotations for variant positions”
ALSYDTEILTVEYGFLPIGKIVEENIECTVYSVDKNGFVYTQPIAQWHNRGEQEVFEYDLEDGSIIRATKDHKFMTTDGEMLPIDEIFEQGLDLKQV

SEQ ID NO: 3 - protein, 35 aa, Unknown
  source: /note=“Description of Unknown C-intein sequence”
VKIVSRKSLGVQNVYDIGVEKDHNFLLANGLIASN

SEQ ID NO: 4 - protein, 284 aa, Artificial Sequence
  source: /note=“Description of Artificial Sequence Synthetic polypeptide”
METDTLLLWVLLLWVPGSTGVKIVSRKSLGVQNVYDIGVEKDHNFLLANGLIASNRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 5 - protein, 230 aa, Artificial Sequence
  source: /note=“Description of Artificial Sequence Synthetic polypeptide”
MSAWSHPQFEKGGGSGGGSGGSAWSHPQFEKGGGSGGGSVKIVSRKSLGVQNVYDIGVEKDHNFLLANGLIASNAFVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSSAAA